(19) United States
(12) Patent Application Publication
(10) Pub. No.: US 2026/0356845 A1
(43) Pub. Date: Nov. 12, 2026

(54) METHOD FOR PREPARING ISOSORBIDE ESTER SULFATE AND COMPOSITIONS THEREOF

(71) Applicants: BASF SE, Ludwigshafen am Rhein (DE); The Procter & Gamble Company, Cincinnati, OH (US)

(72) Inventors: Markus DIERKER, Duesseldorf-Holthausen (DE); Juergen TROPSCH, Ludwigshafen am Rhein (DE); Stephan DOHMEN, Duesseldorf-Holthausen (DE); Frank CLASEN, Duesseldorf-Holthausen (DE); Sophie MAITRO-VOGEL, Ludwigshafen am Rhein (DE); Christian Hubert WEIDL, Ludwigshafen am Rhein (DE); Karl BRAECKMAN, Cincinnati, OH (US); Jan BILLIAUW, Cincinnati, OH (US); Phillip Kyle VINSON, Cincinnati, OH (US)

(21) Appl. No.: 19/583,996

(22) PCT Filed: Oct. 22, 2024
(86) PCT No.: PCT/EP2024/079784

§ 371 Date: May 1, 2026

(30) Foreign Application Priority Data
Nov. 2, 2023 (EP) 23207466.6

(51) Int. Cl. C07D 493/04; C09K 23/32; C11D 1/26

(52) U.S. Cl. CPC C07D 493/04; C09K 23/32; C11D 1/26; C11D 2111/12

(57) ABSTRACT

This invention deals with a modified method for preparing isosorbide ester sulfate and compositions comprising an isosorbide ester sulfate and a fatty alcohol sulfate and the use of such compositions, particularly in cleaning compositions, such as laundry detergent or hand dish compositions or in emulsion polymerization as emulsifier.

This invention deals with a modified method for preparing isosorbide ester sulfate and compositions comprising an isosorbide ester sulfate and a fatty alcohol sulfate and the use of such compositions, particularly in cleaning compositions, such as laundry detergent, hand dish compositions or in emulsion polymerization as emulsifier.

Detergent and home care product formulators are continuously faced with the task of developing improved products to remove a broad spectrum of soils and stains from fabrics and hard surfaces. Chemically and physico-chemically, the varieties of soils and stains spectrum range from polar soils, such as proteinaceous, clay, and inorganic soils, to non-polar soils, such as soot, carbon-black, by-products of incomplete hydrocarbon combustion, and organic soils like sebum and starch-based soils. These challenging demands become even more challenging in view of the emerging trends to improve the “footprint” of any cleaning product, be it in terms of its origin like being from natural or renewable resources, or compared to previous products, its production in terms of production efficiency and thus reduced usage of energy, its efficiency in usage such as reduced amounts for the same performance or higher performance at the same amount levels used, its persistence in the natural environment after its usage, especially its biodegradation, since recycling is technically very challenging and therewith economically not attractive.

As a result of these trends, there is a strong need for improved and more sustainable production processes of surfactants. One step into this direction is the reduction of energy used for the preparation process of the surfactant. A main factor contributing to energy efficiency during the production process of surfactants is the temperature applied during the different synthesis steps.

Regarding surfactants, BASF examined between 2008 and 2011 in a broad screening approach the suitability of various isosorbide derivatives for use in detergents, cleaning agents and cosmetics. As a result, isosorbide esters have proven to be particularly interesting for detergent applications and cosmetic products. Alkoxylates performed well when cleaning plastic surfaces and sulfates showed good foaming performance.

Isosorbide ester sulfates are synthesized starting from isosorbide and a carboxylic acid reacting both substrates in a condensation reaction to form an isosorbide monoester. Subsequently, this intermediate compound is sulfated. As the isosorbide monoester has a relatively high melting point (over 60 °C), two alternative approaches for sulfation have been developed. One approach is directed to a batch process at room temperature using chlorosulfuric acid and tetrahydrofuran (THF) as solvent. However, the batch process is inefficient and chlorosulfuric acid and THF are considered being harmful (GH S07), health hazards (GH S08) and corrosive and their use should therefore be avoided. Alternatively, the sulfation can be carried out using sulfur trioxide, oleum or disulfuric acid at a temperature of 90 to 100 °C using a falling film reactor. These temperatures are required due to the high viscosity of isosorbidester at lower temperatures and the arrangement of pipes in the falling film reactor. However, due to its high temperature, this process is energy insufficient.

In the following, a summary of the current knowledge and most relevant publications in the field of the present invention, the synthesis of isosorbide ester sulfates is given.

EP2270017 B1 discloses in Example 2 the synthesis of an isosorbide ether sulfate. The synthesis protocol involves sulfation at temperatures not exceeding 25 °C based on chlorosulfuric acid and tetrahydrofuran (THF) as solvent. As described above, these compounds are harmful (GH S07), health hazards (GH S08) and corrosive.

Thus, there is a need in the art to find a preparation method for isosorbide ester sulfates that is free of organic solvents, does not involve compounds which are harmful, health hazards or corrosive, that is energy efficient and does not depend on high temperature steps and that is additionally applicable to the falling film reactor technology to provide an efficient synthesis.

Surprisingly, the present inventors found that isosorbide monoester can be combined with fatty alcohols to achieve i) a significantly lower melting point of the mixture compared to the melting point of the sole isosorbide monoester and ii) in parallel a significantly lower viscosity compared to the viscosity of the isosorbide monoester at similar temperatures. The mixture of the isosorbide monoester and fatty alcohol can therefore be sulfated in a reaction that is free of organic solvent and which can be carried out on a falling film reactor. Moreover, the temperature of the sulfation step can be lowered below 98 °C making the process energy efficient. The resulting composition comprising an isosorbide ester sulfate and a fatty alcohol sulfate can be used, for example, as laundry or hand dish detergent compositions.

In addition, it was surprisingly found that the composition comprising an isosorbide ester sulfate and a fatty alcohol sulfate can be used as an emulsifier for emulsion polymerization.

Thus, the object of the present invention is to provide a novel method for preparing isosorbide ester sulfate comprising the steps a) to c) as follows:
a) reaction of i) isosorbide and ii) carboxylic acid comprising at least 10 carbon atoms, in order to obtain a first intermediate (I1);
b) mixing i) the first intermediate (I1) with ii) fatty alcohol comprising at least 10 carbon atoms, in order to obtain a second intermediate mixture (I2), wherein the second intermediate mixture (I2) comprises 35 to 90% by weight of the fatty alcohol; and
c) reaction of i) the second intermediate mixture (I2) with ii) at least one of sulfur trioxide, oleum or disulfuric acid in a falling film reactor in order to obtain the isosorbide ester sulfate.

The term “isosorbide ester sulfate”, as used herein, refers to a group of chemical compounds that is prepared according to the method of the present invention. In preferred embodiments, the isosorbide ester sulfate is a compound according to formula (I):

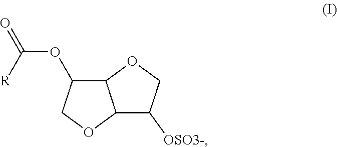

wherein R is linear C₉-C₃₃ alkyl, preferably linear C₁₀-C₂₄ alkyl, more preferably C₁₁-C₁₇ alkyl and even more preferably C₁₁-C₁₃ alkyl. In other preferred embodiments, R is a linear C₁₁, C₁₃, C₁₅ or C₁₇ alkyl or a mixture thereof. Preferred mixtures comprise isosorbide ester sulfates wherein R is a linear C₁₁ and C₁₃ alkyl or R is a linear C₁₅ and C₁₇ alkyl. In other preferred embodiments, the linear alkyl is saturated.

The term “isosorbide”, as used herein, refers to a compound possessing the structure of formula (II). Isosorbide is also known as D-Isosorbide, 1,4:3,6-Dianhydro-D-sorbitol, 1,4-Dianhydrosorbitol and (3R,3aR,6S,6aR)-Hexahydrofuro[3,2-b]furan-3,6-diol.

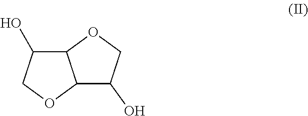

The term “carboxylic acid comprising at least 10 carbon atoms”, as used herein, refers to a linear carboxylic acid according to formula (III), its deprotonated (anionic) form or salts thereof.

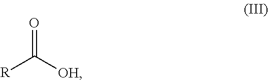

wherein R is defined as described above. For sake of clarity, R is linear C9-C33 alkyl, preferably linear C10-C24 alkyl, more preferably C11-C17 alkyl and even more preferably C11-C13 alkyl. In other preferred embodiments, R is a linear C11, C13, C15 or C17 alkyl or a mixture thereof. Preferred mixtures comprise carboxylic acids wherein R is a linear C11 and C13 alkyl or R is a linear C15 and C17 alkyl. In other preferred embodiments, the linear alkyl is saturated.

The term “first intermediate (I1)”, as used herein, refers to an isosobide monoester. In preferred embodiments, the first intermediate (I1) is a compound according to formula (IV):

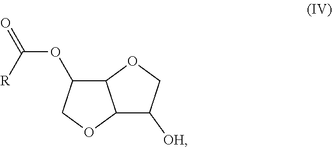

wherein R is linear C9-C33 alkyl, preferably linear C10-C24 alkyl, more preferably C11-C17 alkyl and even more preferably C11-C13 alkyl. In other preferred embodiments, R is a linear C11, C13, C15 or C17 alkyl or a mixture thereof. Preferred mixtures comprise isosorbide ester sulfates wherein R is a linear C11 and C13 alkyl or R is a linear C15 and C17 alkyl. In other preferred embodiments, the linear alkyl is saturated.

The reaction to obtain the first intermediate (I1) is a condensation reaction comprising the reaction of isosorbide and the above-described carboxylic acid comprising at least 10 carbon atoms. In detail, the conditions of this reaction comprise application of several different temperatures including the application of temperatures up to 225 °C. Moreover, in one embodiment a catalysator may be used for said reaction. The catalysator may be Tin (II) oxalate. Alternatively, the skilled person is aware of similar catalysators that can be used for the reaction to obtain the first intermediate (I1). In other embodiments, the yield of the reaction is at least 60%, at least 65%, at least 70%, at least 75%, at least 80% or at least 85% of the theoretical yield.

The term “fatty alcohol comprising at least 10 carbon atoms”, as used herein, refers to a primary alcohol. In preferred embodiments, the fatty alcohol is a primary, monovalent and saturated fatty alcohol. Alternatively, fatty alcohol is a compound according to formula (V):

H₃C—(CH₂)ₙ—CH₂—OH (V),

wherein n is at least 8, at least 9 or at least 10. In preferred embodiments, n is an integer ranging from 8 to 31, more preferred from 9 to 22, even more preferably from 10 to 16 and most preferred from 10 to 12. In other preferred embodiments, n is 10, 12, 14 or 16 or a mixture thereof. Preferred mixtures comprise fatty alcohols wherein n is 10 and 12 or n is 14 and 16.

The term “second intermediate mixture (I2)”, as used herein, refers to a mixture of (i) the above-described first intermediate (I1)/isosorbide monoester and (ii) the above-described fatty alcohol comprising at least 10 carbon atoms. It is noted that the first intermediate (I1) as well as the fatty alcohol may be mixtures by itself. This means that each of the first intermediate (I1) and the fatty alcohol can comprise a heterogenic group of compounds as described above. Thus, for example the second intermediate mixture (I2) may comprise isosorbide monoester of formula (IV), wherein R is linear C11 and C13 alkyl and fatty alcohol of formula (V), wherein n is 10 and 12. In other preferred embodiments, the melting point of the second intermediate mixture (I2) is below 70 °C, below 65 °C, below 60 °C, below 55 °C, below 50 °C, below 45 °C, below 40 °C or below 37 °C.

The second intermediate mixture (I2) comprises 35 to 90% by weight of the fatty alcohol. In preferred embodiments, the second intermediate mixture (I2) comprises 40 to 90%, 45 to 85%, 50 to 80%, 55 to 75% or 60 to 70% by weight of the fatty alcohol.

The term “sulfur trioxide”, as used herein, refers to a compound of the formula SO₃ or salts thereof. Synonyms are sulfonylideneoxidane, sulfuric anhydride and sulfur(VI) oxide.

The term “disulfuric acid”, as used herein, refers to a compound of formula (VI), its depronated forms or salts thereof:

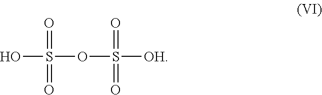

The term “oleum”, as used herein, refers to solutions of various compositions of sulfur trioxide in sulfuric acid (H₂SO₄), or sometimes more specifically to disulfuric acid (also known as pyrosulfuric acid and shown in formula (VI)). Oleum is identified by the CAS number 8014-95-7.

The final reaction to obtain the isosorbide ester sulfate is a sulfation reaction comprising the reaction of the above-described second intermediate mixture (I2) and sulfur trioxide, oleum or disulfuric acid in a falling film reactor. The reaction temperature may be not higher than 98 °C, not higher than 90 °C, not higher than 80 °C, not higher than 75 °C, not higher than 70 °C, not higher than 65 °C, not higher than 60 °C, not higher than 55 °C, not higher than 50 °C, not higher than 45 °C, not higher than 40 °C or not higher than 37 °C.

Sulfation may be carried out continuously, especially in reactors that operate according to the falling film principle. Preference is given to the reaction in the absence of solvents. Specifically, the conditions of this reaction include the reaction with gaseous sulfur trioxide in a mixture with inert gas. Sulfur trioxide is diluted with air or nitrogen and preferably used in the form of a gas mixture with approx. 1% to 7%, in particular 3% to 5% sulfur trioxide. The reaction can be carried out with a molar ratio of isosorbide ester/fatty alcohol mixture to sulfur trioxide of 1:0.9 to 1:1.3. Sulfation can be carried out at temperatures ranging from 50 to 98 °C. To ensure a sufficiently low viscosity of the feedstocks and, on the same time, avoid excessive thermal stress during the reaction, it is recommended to carry out sulfation between 50 °C and 80 °C.

The acidic sulfation products resulting from the reaction of the isosorbide ester/fatty alcohol mixture with sulfur trioxide may then be neutralized by combining the sulfide products and an aqueous base and maintaining a pH value of 6.0 to 7.5 with the addition of suitable buffers (for example KH₂PO₄, NaH₂PO₄). Alkali metal hydroxides such as sodium hydroxide and potassium hydroxide can be considered as bases for neutralization.

Step c) is conducted in a falling film reactor. Such falling film reactors and their use for sulf(on)ation are well-known in the art and include Stepan's Falling Film Reactor (for example, as described in U.S. Pat. No. 3,169,142A) and Chemithon's Annular Film Reactor (for example, as described in U.S. Pat. No. 3,427,342A).

In further embodiments, the carboxylic acid comprising at least 10 carbon atoms
is linear;
has as the only functional group one carboxyl group; and/or
is a C12 or C14 carboxylic acid or a mixture thereof.

“Linear” carboxylic acid means that, aside the carbon atom of the carboxyl group, the compound only contains one terminal primary carbon atom and the rest of the carbon atoms are secondary carbon atoms attached to two other carbon atoms.

The term “only functional group (is) one carboxyl group”, as used herein, describes that the only functional group in the compound is the carboxyl group, whereas other groups, such as further carboxyl groups, alcohol groups, carbonyl groups, hetero atomic groups etc. are absent.

In further embodiments, the fatty alcohol comprising at least 10 carbon atoms
is linear;
has as the only functional group one primary alcohol group; and/or
is a C12, C14, C16 or C18 fatty alcohol or a mixture thereof, preferably a mixture of C12 and C14 fatty alcohols or a mixture of C16 and C18 fatty alcohols.

The terms “linear” is as defined for the carboxylic acid. The term “only functional group (is) one primary alcohol group”, as used herein, describes that the only functional group in the compound is the primary alcohol group, whereas other groups, such as further alcohol groups, carboxylic groups, carbonyl groups, hetero atomic groups etc. are absent.

In further embodiments, the second intermediate mixture (I2) comprises 40 to 85%, preferably 50 to 80%, more preferably 60 to 75% by weight of the fatty alcohol or its mixtures.

In further embodiments, in step c) of the inventive method the second intermediate mixture (I2) is reacted with sulfur trioxide.

In further embodiments, the temperature in step c) of the inventive method is not higher than 98 °C, not higher than 90 °C, not higher than 80 °C, not higher than 75 °C, not higher than 70 °C, not higher than 65 °C, preferably not higher than 60 °C or more preferably not higher than 55 °C.

In further embodiments, in step a) of the inventive method the molar ratio of isosorbide to carboxylic acid is at least 1.5:1, preferably at least 4:1 or more preferably at least 6:1. Further, in step a) of the inventive method the molar ratio of isosorbide to carboxylic acid is not higher than 20:1, preferably not higher than 15:1 or more preferably not higher than 10:1.

In further embodiments, the first intermediate (I1) is purified by distillation. After this purification, the first intermediate (I1) has a purity level of at least 50%, at least 60%, at least 70%, at least 80%, at least 90%, at least 95%, at least 99% or at least 99.5% based on the mole fraction (i.e. amount of the first intermediate (I1) expressed in moles divided by the total amount of all constituents in the mixture also expressed in moles). In particular, the purification step is carried out to isolate the first intermediate (I1) from non-reacted substrates isosorbide and carboxylic acid and also from isosorbide di-esters (i.e. an isosorbide, wherein each of the two alcohol groups reacted with a carboxylic acid).

In addition, the isosorbide ester sulfate may be further submitted to the following process steps of
a) purification using standard means such as steam distillation, thermal distillation, vacuum evaporation, including removal of all solvent, dialysis and/or
b) drying using standard drying means such as spray-, drum, paddle-, vacuum-drying means including agglomeration methods such as fluidized-bed-drying,
   to obtain a purified isosorbide ester sulfate solution, a purified isosorbide ester sulfate liquid, a solid, e.g., granulated or co-granulated isosorbide ester sulfate or a purified solid isosorbide ester sulfate, respectively.

In case that after the reaction leading to the isosorbide ester sulfate residual substrates are present to a non-desirable extent, the resulting product mixture containing the isosorbide ester sulfate may be further purified by standard means to reduce the content of residual substrates, but also to reduce the amount of possible by-products, reduce the amount(s) of the solvent(s) employed (i.e., to concentrate) or replace solvent(s) with other solvents. Such processes are known to a person of skill in this field.

Preferably, undesirable amounts of residual substrates are removed, preferably by means of distillative processes, more preferably by thermal distillative processes, which may additionally comprise the application of reduced pressure to increase the speed and/or the effectiveness of the removal.

In a preferred embodiment only the additional process step a) is employed.

In further embodiments, at least one of the isosorbide, carboxylic acid and fatty alcohol is a renewable compound, preferably isolated from an organism such as bacteria, fungi or plants. In preferred embodiments, all of the above-mentioned three substrates are renewable compounds. Thus, the isosorbide can be produced from sorbitol. Sorbitol is produced from (corn) starch as the raw material using enzymatic liquefaction and a saccharification process to generate glucose which then is concentrated and hydrogenated to produce sorbitol. Natural based and renewable isosorbide is commercially available from Roquette Freres, Lestrem, France and is sold as POLYSORB®.

Long-chained carboxylic acids are well-known metabolites of bacteria, fungi or plants. These compounds are therefore naturally based and renewable. Several different protocols have been developed for their isolation and thus such carboxylic acids are frequently available on the market. Moreover, these carboxylic acids can also to produce fatty alcohols (a) directly by hydrogenation or (b) indirectly by forming a methyl ester which is subsequently hydrogenated to a fatty alcohol. Therefore, the skilled person also knows how naturally based and renewable fatty alcohols can be synthesized.

In a second aspect, the present invention is directed to the use of fatty alcohol comprising at least 10 carbon atoms during sulfation of isosorbide ester, preferably for lowering the melting temperature and/or viscosity of the isosorbide ester, wherein the isosorbide ester comprises an alkyl group possessing at least 10 carbon atoms, wherein the sulfation is preferably conducted in a falling film reactor.

For all the terms within the second aspect that have already been defined and explained in detail herein before within the description of the first aspect (i.e. the method for preparing an isosorbide ester sulfate), such terms and definitions of course apply to the second aspect.

In a preferred embodiment, the use of fatty alcohol comprising at least 10 carbon atoms during sulfation of isosorbide ester is carried out using a falling film reactor. Such falling film reactors and their use for sulf(on)ation are well-known in the art and include Stepan's Falling Film Reactor (for example, as described in U.S. Pat. No. 3,169,142A) and Chemithon's Annular Film Reactor (for example, as described in U.S. Pat. No. 3,427,342A).

In a third aspect, the present invention is directed to a composition comprising an isosorbide ester sulfate and a fatty alcohol sulfate, wherein the isosorbide ester sulfate and the fatty alcohol sulfate each comprise an alkyl group possessing of at least 10 carbon atoms and at least 40% of the combined weight of the isosorbide ester sulfate and the fatty alcohol sulfate is based on the fatty alcohol sulfate.

For all the terms within the third aspect that have already been defined and explained in detail herein before within the description of the first and second aspect (i.e. the method for preparing an isosorbide ester sulfate and use of fatty alcohol during sulfation of isosorbide ester), such terms and definitions of course apply to the third aspect.

The term “fatty alcohol sulfate”, as used herein, refers to a compound according to formula (VII), its deprotonated (anionic) form or salts thereof.

$${H}_{3} C - {( {CH}_{2} )}_{n} - {CH}_{2} - {OSO}_{3} H , \qquad ( VII )$$

wherein n is at least 8, at least 9 or at least 10. In preferred embodiments, n is an integer ranging from 8 to 31, more preferred from 9 to 22, even more preferably from 10 to 16 and most preferred from 10 to 12. In other preferred embodiments, n is 10, 12, 14 or 16 or a mixture thereof. Preferred mixtures comprise fatty alcohol sulfates wherein n is 10 and 12 or n is a 14 and 16.

The term “alkyl group of the isosorbide ester sulfate”, as used herein, refers to the group represented as R in formula (I). The term “alkyl group of the fatty alcohol sulfate”, as used herein, refers to the group represented by the part H₃C(CH₂)ₙCH₂— of formula (VII), wherein the dotted line indicates the bond to the sulfation group.

In further embodiments, the alkyl group of the isosorbide ester sulfate
is linear; and/or
is a C12 or C14 alkyl group or the composition comprises a mixture of C12 and C14 isosorbide ester sulfates.

In further embodiments, the fatty alcohol sulfate
is linear; and/or
is a C12, C14, C16 or C18 fatty alcohol sulfate or a mixture thereof, preferably a mixture of C12 and C14 fatty alcohol sulfates or a mixture of C16 and C18 fatty alcohol sulfates.

In further embodiments, at least 45%, preferably at least 50%, more preferably at least 60% of the combined weight of the isosorbide ester sulfate and the fatty alcohol sulfate is based on the fatty alcohol sulfate or its mixtures. In other embodiments, not more than 90%, preferably not more than 80%, more preferably not more than 70% of the combined weight of the isosorbide ester sulfate and the fatty alcohol sulfate is based on the fatty alcohol sulfate or its mixtures.

In alternative embodiments, the molar ratio in the composition of the invention between the isosorbide ester sulfate and the fatty alcohol sulfate ranges from 1:0.5 to 1:10, preferably 1:1 to 1:7, more preferably 1:1.5 to 1:5 and even more preferably 1:2 to 1:3.

In further embodiments, the inventive composition is selected from the group consisting of cleaning composition, fabric and home care product, industrial and institutional cleaning product, cosmetic formulation, emulsifier for emulsion polymerization, crude oil emulsion breaker, pigment dispersion for ink jet inks, formulation for electro plating, cementitious composition, dispersant for agrochemical formulations, preferably cleaning composition and/or fabric and home care product and/or industrial and institutional cleaning product.

The inventive composition can be added to cosmetic formulations, as crude oil emulsion breaker, as an emulsifier in emulsion polymerization, in pigment dispersions for ink jet inks, formulations for electro plating, in cementitious compositions. However, the inventive composition can also be added to (used in) washing or cleaning compositions.

Another subject-matter of the present invention is, therefore, a cleaning composition, fabric and home care product, industrial and institutional cleaning product, cosmetic formulation, crude oil emulsion breaker, pigment dispersion for inkjet inks, formulation for electro plating, cementitious composition and/or dispersant for agrochemical formulations, comprising at least one isosorbide ester sulfate and at least one fatty alcohol sulfate, each as defined above.

Preferably, it is a cleaning composition and/or fabric and home care product, comprising the inventive composition as defined above, preferably for improved clay removal or oily and fatty stain removal, preferably a laundry detergent formulation and/or a manual dish wash detergent formulation, more preferably a liquid laundry detergent formulation and/or a liquid manual dish wash detergent formulation.

In another preferred embodiment of the present invention, the cleaning composition may be used for soil removal of particulate stains and/or oily and fatty stains, and additionally for whiteness maintenance, preferably in laundry care.

In another embodiment, the cleaning composition of the present invention is a hard surface cleaning composition that may be used for cleaning various surfaces such as hard wood, tile, ceramic, plastic, leather, metal, glass.

In another embodiment, the cleaning composition of the present invention is a liquid or solid automatic dish wash detergent composition, preferably a solid automatic dish wash detergent composition, that may be used for cleaning dish ware, e.g., dish ware such as glasses, wherein the inventive composition is improving the removal of stubborn soils.

In another embodiment, the cleaning composition is designed to be used in personal care and pet care compositions such as shampoo compositions, body wash formulations, liquid or solid soaps.

In this invention, a preferred area of application for the use of the inventive composition is the field of fabric and home care products and cleaning compositions, preferably cleaning compositions for industrial and institutional use and the use by consumers in their household.

Moreover, the inventive composition further comprises
a) at least one element of the group consisting of non-anionic surfactants, amphoteric surfactants, builders, chelating agents, co-builders, alcohols, thickeners, water soluble polymers, clay soil removal/anti-redeposition agents, polymeric soil release agents, bleaching agents, bleach activators, brighteners, malodor control agents, pigments, dyes, opacifiers, hueing agents, dye transfer inhibiting agents, suds suppressors (antifoams), anti-corrosion agents, softeners and perfumes;
b) an antimicrobial agent selected from the group consisting of 2-phenoxyethanol and 4,4′-dichloro 2-hydroxydiphenylether; and/or
c) at least one enzyme selected from the list consisting of lipases, hydrolases, amylases, DNases, proteases, cellulases, hemicellulases, phospholipases, esterases, mannanases, xylanases, dispersins, oxidoreductases, cutinases, pectate lyases, pectinases, lactases and peroxidases, and combinations of at least two of the foregoing types, preferably selected from one or more lipases, hydrolases, amylases, proteases, cellulases, and combinations of at least two of the foregoing types, more preferably at least one enzyme being selected from proteases.

In a fourth aspect, the present invention is directed to a use of the inventive composition,
A) preferably a cleaning composition and/or fabric and home care product, for
i) improved removal of oily/fatty stains, and/or
ii) clay removal, and/or
iii) soil removal of particulate stains, and/or
iv) dispersion and/or emulsification of soils, and/or
v) modification of treated surface to improve removal upon later re-soiling, and/or
vi) whiteness improvement and/or
most preferably in cleaning compositions for
i) removal of oily/fatty stains and/or ii) clay removal,
each of the before mentioned options i) to vi) preferably for use in a laundry detergent formulation and/or a manual dish wash detergent formulation and/or in a formulation suitable for (pre)-treatment of textiles and/or soap bars, more preferably in a liquid laundry detergent formulation and/or a liquid manual dish wash detergent formulation; or
B) preferably as an emulsifier in a polymerization reaction.

In another aspect, the presently claimed composition is used in a method of emulsion polymerization comprising the steps of
1. charging into a reactor a reaction mixture comprising:
a. at least one monomer having an ethylenically unsaturated double bond,
b. the inventive composition, and
c. at least one polymerization initiator; and
2. maintaining the reaction mixture of step (1) in the reactor at a temperature in the range of ≥20 °C to ≤105 °C for a time period in the range of ≥30 minutes to ≤12 hours.

The emulsion polymerization can be a batch process (batchwise), a semi continuous or fully continuous process, such as a feed process.

Suitable reactors include, but are not limited to, continuous stirred tank reactors (CSTRs), tube reactors, loop reactors, extruder reactors, or any reactor suitable for a continuous operation.

A form of CSTR, is a tank reactor provided with cooling coils and/or cooling jackets. The cooling coils and/or the cooling jackets have to be sufficient to remove any heat of the polymerization not taken up by raising the temperature of the continuously charged monomer composition to maintain a preselected temperature for polymerization therein. Such a CSTR may be provided with at least one, and usually more, agitators to provide a well-mixed reaction zone. Such CSTR may be operated at varying filling levels from 20 to 100% full (liquid full reactor LFR). In one embodiment the reactor is more than 50% full, but less than 100% full. In another embodiment the reactor is 100% liquid full.

In a preferred embodiment, the temperature in step (2) is in the range of ≥40 °C to ≤105 °C or ≥40 °C to ≤100 °C or ≥40 °C to ≤95 °C or ≥40 °C to ≤85 °C, more preferably in the range of ≥50 °C to ≤105 °C or ≥50 °C to ≤100 °C or ≥50 °C to ≤95 °C or ≥50 °C to ≤85 °C, even more preferably in the range of 60 °C to ≤105 °C or ≥60 °C to ≤100 °C or ≥60 °C to ≤95 °C or ≥60 °C to ≤90 °C.

The most favorable temperature chosen is dependent on the decomposition characteristics of the initiator used or of the initiators used.

The pressure conditions are generally not critical and, for example, pressures in the range from atmospheric pressure to 10 bar are suitable.

In a preferred embodiment, the time period of the emulsion polymerization is in the range from ≥1 hour to ≤10 hours or ≥1 hour to ≤9 hours or ≥1 hour to ≤8 hours or ≥1 hour to ≤7 hours or ≥1 hour to ≤6 hours, more preferably in the range of ≥2 hours to ≤10 hours or ≥2 hours to ≤9 hours or ≥2 hours to ≤8 hours or ≥2 hours to ≤7 hours or ≥2 hours to ≤6 hours or ≥2 hours to ≤5 hours.

The steps (1) and (2) can preferably be carried out in the presence of an inert gas, such as nitrogen or argon.

In an embodiment, the reaction mixture of the process of the presently claimed invention comprises at least one solvent.

The at least one monomer having an ethylenically unsaturated double bond is selected from the group consisting of
acrylic monomers;
vinyl aromatic hydrocarbons; and
vinyl, allyl, and methallyl esters of saturated aliphatic C₂-C₂₄ monocarboxylic acids.

In a preferred embodiment of the presently claimed invention, the at least one monomer having an ethylenically unsaturated double bond is an acrylic monomer.

In another preferred embodiment of the presently claimed invention, the acrylic monomer is a (meth)acrylic monomer.

As used herein, “(meth)acrylic monomer” refers to acrylic acid or methacrylic acid, esters of acrylic or methacrylic acid, salts, amides, and other suitable derivatives of acrylic or methacrylic acid and mixtures thereof. Examples of suitable acrylic monomers include, without limitation, the following methacrylate esters: methyl (meth)acrylate, ethyl (meth)acrylate, n-propyl (meth)acrylate, n-butyl (meth)acrylate, isopropyl (meth)acrylate, isobutyl (meth)acrylate, n-amyl (meth)acrylate, n-hexyl (meth)acrylate, isoamyl (meth)acrylate, 2-hydroxyethyl (meth)acrylate, 2-hydroxypropyl (meth)acrylate, N,N-dimethylaminoethyl (meth)acrylate, N,N-diethylaminoethyl (meth)acrylate, t-butylaminoethyl (meth)acrylate, trifluoroethyl (meth)acrylate, glycidyl (meth)acrylate, benzyl (meth)acrylate, allyl (meth)acrylate, 2-n-butoxyethyl (meth)acrylate, 2-chloroethyl (meth)acrylate, sec-butyl-(meth)acrylate, tert-butyl (meth)acrylate, 2-ethylbutyl (meth)acrylate, cinnamyl (meth)acrylate, crotyl (meth)acrylate, cyclohexyl (meth)acrylate, cyclopentyl (meth)acrylate, 2-ethoxyethyl (meth)acrylate, furfuryl (meth)acrylate, hexafluoroisopropyl (meth)acrylate, methallyl (meth)acrylate, 3-methoxybutyl (meth)acrylate, 2-methoxybutyl (meth)acrylate, 2-nitro-2-methylpropyl (meth)acrylate, n-octyl(meth)acrylate, 2-ethylhexyl (meth)acrylate, 2-phenoxyethyl (meth)acrylate, 2-phenylethyl (meth)acrylate, phenyl(meth)acrylate, propargyl (meth)acrylate, tetrahydrofurfuryl (meth)acrylate and tetrahydropyranyl (meth)acrylate. Example of suitable acrylate esters include, without limitation, methyl acrylate, ethyl acrylate, n-propyl acrylate, isopropyl acrylate, n-butyl acrylate and n-decyl acrylate.

Examples of other suitable acrylic monomers include, without limitation, methacrylic acid derivatives such as: methacrylic acid and its salts, methacrylonitrile, methacrylamide, N-methylmethacrylamide, N-ethylmethacrylamide, N,Ndiethylmethacrylamide, N,N-dimethylmethacrylamide, N-phenylmethacrylamide and methacrolein. Examples of acrylic acid derivatives include, without limitation, acrylic acid and its salts, acrylonitrile, acrylamide, methyl α-chloroacrylate, methyl 2-cyanoacrylate, N-ethylacrylamide, N,N-diethylacrylamide and acrolein.

Preferably, the at least one acrylic monomer is selected from the group consisting of ethyl acrylate, methyl (meth)acrylate, butyl(meth)acrylate, 2-ethylhexyl(meth)acrylate, hydroxyethyl(meth)acrylate and acrylic acid.

More preferably, the (meth)acrylic monomer is selected from the group consisting of methyl (meth)acrylate, butyl(meth)acrylate, n-butyl acrylate, and acrylic acid.

In a preferred embodiment of the presently claimed invention, the vinyl aromatic hydrocarbon is selected from the group consisting of styrene, vinyltoluene, tert-butylstyrene and α-methylstyrene. More preferably, the vinyl aromatic hydrocarbon is styrene.

In a preferred embodiment, the at least one monomer having an ethylenically unsaturated double bond is selected from the group consisting of vinyl, allyl and methallyl esters of saturated aliphatic C₂-C₂₄ monocarboxylic acids.

In a preferred embodiment, the at least one monomer having an ethylenically unsaturated double bond is a vinyl ester of a saturated aliphatic C₂-C₂₄ monocarboxylic acid.

In another preferred embodiment, the vinyl ester of a saturated aliphatic C₂-C₂₄ monocarboxylic acid is selected from the group consisting of vinyl acetate, vinyl propionate, vinyl butyrate, vinyl pivalate, vinyl hexanoate, vinyl-2-ethylhexanoate, vinyl laurate and vinyl stearate.

In a preferred embodiment, the amount of the at least one monomer having an ethylenically unsaturated double bond is in the range of 35% to 80% by weight, based on the total weight of the reaction mixture.

### Polymerization Initiator

The emulsion polymerization is affected using at least one initiator. The at least one polymerization initiator suitable for carrying out the process according to the presently claimed invention may thermally decompose into radicals in a first order reaction.

In a preferred embodiment, the at least one initiator is an inorganic free radical initiator.

In a preferred embodiment, the inorganic free radical initiator is selected from the group consisting of ammonium persulfate, potassium persulfate, sodium persulfate, potassium peroxy dicarbonate, potassium peroxy diphosphate and mixtures thereof.

The at least one initiator can be selected from the group consisting of peroxides or systems containing at least one peroxide, such as redox initiators containing at least one peroxide. Examples of suitable peroxides are alkali metal peroxodisulfates, such as, for example, sodium peroxodisulfate or ammonium peroxodisulfate, hydrogen peroxide, organic peroxides, such as diacetyl peroxide, di-tert-butyl peroxide, diamyl peroxide, dioctanoyl peroxide, didecanoyl peroxide, dilauryl peroxide, dibenzoyl peroxide, bis(o-toluyl) peroxide, succinyl peroxide, tert-butyl peracetate, tert-butyl permaleate, tert-butyl perisobutyrate, tert-butyl perpivalate, tert-butyl peroctanoate, tert-butyl pemeodecanoate, tert-butyl perbenzoate, di-tertbutyl peroxide, tertbutyl hydroperoxide, cumyl hydroperoxide, tert-butyl peroxy-2-ethylhexanoate and diisopropyl peroxydicarbamate. Azo compounds, such as, for example, azobisisobutyronitrile, azobis(2-amidopropane) dihydrochloride and 2,2′-azobis(2-methylbutyronitrile) are also suitable. Redox initiators are likewise suitable, for example comprising peroxides or an oxidizable sulfur compound. Examples are combinations of at least one Fe(II) compound such as FeSO₄ and at least one peroxide such as H₂O₂, combinations of sodium persulfate and sodium metabisulfite, combinations of organic hydroperoxides and sodium metabisulfite, and combinations of tertiary amines and dibenzoyl peroxide.

The amount of polymerization initiator depends upon the conditions of the reaction and may be adjusted accordingly.

In a preferred embodiment, the at least one polymerization initiator is present in an amount in the range of ≥0.1% to ≤5%, preferably in the range of ≥0.1% to ≤4.5% or ≥0.1% to ≤4%, more preferably in the range of ≥0.1% to ≤5% or ≥0.1% to ≤4.5% or ≥0.1% to ≤4.0%, and most preferably in the range of ≥0.2% to ≤4.5% or ≥0.2% to ≤4.0% or ≥0.2% to ≤3.5% or ≥0.2% to ≤3.0% by weight, in each case based on the total weight of the reaction mixture.

In a preferred embodiment, the step (C) is carried out with a terminating compound selected from the group consisting of sodium bisulfite and sodium metabisulfite.

### Solvent

In a preferred embodiment, the reaction mixture of the process of the presently claimed invention comprises at least one solvent.

The at least one solvent may be fed into the reactor together with the at least one monomer having an ethylenically unsaturated double bond, i.e. the at least one solvent and the at least one monomer having an ethylenically unsaturated double bond are fed in one single feed, or it may be fed into the reactor separately, i.e. the at least one solvent and the at least one monomer having an unsaturated double bond are fed in two feeds.

In a preferred embodiment, the at least one solvent is selected from the group comprising of water, xylene, toluene, ethyl benzene, light naphtha, heavy naphtha, acetone, methylethylketone, methylamylketone, methyl iso-butylketone, N-methylpyrrolidone, isopropanol, propylene glycol monomethyl ether acetate, acetic acid, propyl acetate, dipropylene glycol methyl ether acetate, propylene glycol diacetate, dipropylene glycol dimethyl ether, ethyl 3-ethoxy propionate and isoparrafin.

In a preferred embodiment, the at least one solvent is water.

The at least one solvent is present in an amount in the range of ≥15% to ≤60% or ≥15% to ≤59% or ≥15% to ≤58% or ≥15% to ≤57% or ≥15% to ≤56% or ≥15% to ≤55% or ≥15% to ≤54% or ≥15% to ≤53% or ≥15% to ≤52% or ≥15% to ≤51% or ≥15% to ≤50% or ≥15% to ≤49% or ≥15% to ≤48% or ≥15% to ≤47% or ≥15% to ≤46% or ≥15% to ≤45% or ≥15% to ≤44% or ≥15% to ≤43% or ≥15% to ≤42% or ≥15% to ≤41% or ≥15% to ≤40% or ≥15% to ≤39% or ≥15% to ≤38% or ≥15% to ≤37% or ≥15% to ≤36% or ≥15% to ≤35%, more preferably in the range of ≥20% to ≤60% or ≥20% to ≤59% or ≥20% to ≤58% or ≥20% to ≤57% or ≥20% to ≤56% or ≥20% to ≤55% or ≥20% to ≤54% or ≥20% to ≤53% or ≥20% to ≤52% or ≥20% to ≤51% or ≥20% to ≤50% or ≥20% to ≤49% or ≥20% to ≤48% or ≥20% to ≤47% or ≥20% to ≤46% or ≥20% to ≤45% or ≥20% to ≤44% or ≥20% to ≤43% or ≥20% to ≤42% or ≥20% to ≤41% or ≥20% to ≤40% or ≥20% to ≤39% or ≥20% to ≤38% or ≥20% to ≤37% or ≥20% to ≤36% or ≥20% to ≤35% by weight, in each case based on the total weight of the reaction mixture.

In another aspect, the presently claimed invention is directed to a mixture comprising
1. at least one monomer having an ethylenically unsaturated double bond selected from the group consisting of acrylic monomers, vinyl aromatic hydrocarbons and vinyl, allyl and methallyl esters of saturated aliphatic C₂-C₂₄ monocarboxylic acids,
2. an inventive composition as described above, and
3. water.

In an embodiment, the at least one monomer is selected from the group consisting of acrylic acid, methacrylic acid, methyl (meth)acrylate, butyl(meth)acrylate, n-butyl acrylate, styrene, vinyltoluene, tert-butylstyrene and α-methylstyrene, vinyl acetate, vinyl propionate, vinyl butyrate, vinyl pivalate, vinyl hexanoate, vinyl-2-ethylhexanoate, vinyl laurate and vinyl stearate.

In another aspect, the presently claimed invention is directed to a mixture comprising
1. an inventive composition as described above,
2. a polymer P, wherein the polymer P comprises at least one monomer selected from the group consisting of acrylic monomers, vinyl aromatic hydrocarbons and vinyl, allyl and methallyl esters of saturated aliphatic C₂-C₂₄ monocarboxylic acids, in polymerized form, and
3. water.

In an embodiment, the at least one monomer is selected from the group consisting of acrylic acid, methacrylic acid, methyl (meth)acrylate, butyl(meth)acrylate, n-butyl acrylate, styrene, vinyltoluene, tert-butylstyrene, α-methylstyrene, vinyl acetate, vinyl propionate, vinyl butyrate, vinyl pivalate, vinyl hexanoate, vinyl-2-ethylhexanoate, vinyl laurate and vinyl stearate.

For all the terms within the fourth aspect that have already been defined and explained in detail herein before within the description of the first, second and third aspect, such terms and definitions of course apply to the fourth aspect.

In further embodiments, the composition of the invention is used or is furnished to be used in low temperature washing processes and/or low water consumption processes. In this regard, the term “low temperature washing”, as used herein, means that the temperature of the wash liquor, optionally, at a given time t1 is preferably between 20 and 45 °C, more preferably between 30 and 40 °C and in particular between 32 and 35 °C and at another given time t2 is preferably between 30 and 55 °C, more preferably between 35 and 50 °C and in particular between 40 and 45 °C. In a particularly preferred embodiment of the washing process, the temperature of the wash liquor at time t2 is above the temperature of the rinse liquor at time t1. t1 refers to a pre-rinse which is optional. A corresponding temperature curve, in which the temperature of the wash liquor at time t2 is above the temperature of the rinse liquor at time t1, has proven to be superior with regard to the cleaning and final rinse performance.

The term “low water consumption”, as used herein, refers to the overall water consumption of a cleaning process, wherein not more than 80%, not more than 70%, not more than 60%, not more than 50%, not more than 40% or even not more than 30% of the water volume is used in a process compared to a standard process defined by the manufacturer of the cleaning apparatus (i.e. washing machine or automatic dish washer) or the manufacturer of the cleaning agent. Modern cleaning apparatus usually comprise a washing program labelled as “eco”, “green” or “sustainable”. Such programs qualify as “low water consumption” in the sense of the present application.

Compositions of the invention comprise at least one isosorbide ester sulfate and at least one fatty alcohol sulfate as described herein. The inventive composition may be part of another composition, preferably a cleaning composition. The inventive composition should be present in effective amounts. The terms “effective amount”, “effective concentration” or “effective dosage” are defined herein as the amount, concentration or dosage of the inventive composition to achieve significant effects in removal of oily/fatty stains and/or clay removal and/or whitening laundry. In preferred embodiments, the cleaning composition comprises 0.001-10% by weight composition of the invention.

The cleaning compositions can be solid, semi-solid or gel-like, liquid (including spray) or an aerosol. They can be formulated in all types usual for the respective application, such as bars, powders, granulates, agglomerates, pastes, gels, solutions, emulsions, suspensions, etc. They can also be formulated as liquid composition imbibed in wipes or pads.

The cleaning compositions generally contain a carrier. In liquid, semi-solid or gel-like compositions, the carrier is or comprises a solvent, mostly water, (and/or) an alkanol (generally a C2-C3-alkanol, i.e. ethanol, n-propanol and/or isopropanol; these generally also act as wetting agents to allow a better wetting or penetration of the treated substrate by the composition; this latter effect is particularly useful if no surfactant is contained in the composition), an organic solvent different therefrom (details to such further solvents are given below in context with preferred embodiments of the composition) or a mixture thereof. In solid, semi-solid or gel-like compositions, the carrier is or comprises a solid carrier. In solid soaps, the soap component (e.g. the solid salt of long-chained fatty acids) is generally also the carrier.

Depending on the targeted use, the cleaning compositions generally comprise further components. Examples are pH adjusting agents, sequestrants, thickeners, antifreezing agents, antifoaming agents, colorants, or perfumes. Further details to such further components are given below in context with preferred embodiments of the cleaning composition.

In a preferred embodiment, the (liquid) cleaning composition comprises
(a) 0 to 2% by weight (=2 to 90<100 ppm), relative to the total weight of the composition, of the at least one preservative agent;
(b) 0.01-10% by weight, relative to the total weight of the composition of the inventive composition;
(c) 0 to 10% by weight, relative to the total weight of the composition, of one or more (anionic and/or nonionic) surfactants:
(d) 0 to 90% by weight, relative to the total weight of the composition, of at least one C₂-C₃-alkanol;
(e) 0 to 10% by weight, relative to the total weight of the composition, of at least one organic solvent;
(f) 0 to 10% by weight, relative to the total weight of the composition, of at least one sequestrant;
(g) 0 to 10% by weight, relative to the total weight of the composition, of a further additive;
(h) 0 to 5% by weight, relative to the total weight of the composition, of at least one enzyme; and/or
(i) ad 100% by weight, but at least 30% by weight, relative to the total weight of the composition, of water.

Preferably, at least one of components (c) to (h) is present. More preferably, at least component (c) is present.

Additional surfactants (or surface-active compounds) (termed component (c) in the above and below embodiments) can be anionic, cationic, non-ionic or amphoteric (zwitterionic). Anionic, cationic, non-ionic and amphoteric surfactants are widely known in the art.

Anionic surfactants are, for example, of the sulfate, sulfonate or carboxylate type or mixed forms thereof. Examples are
alkyl sulfates (generally of the formula R—O—SO₃⁻M⁺, where R is a long-chained alkyl group, e.g. C₈-C₂₄-alkyl, and M⁺ is a cation equivalent, generally Na⁺, K⁺, NH4⁺, mono-, di- or triethanolammonium); e.g. sodium lauryl sulfate;
alkyl ether sulfates (generally of the formula R—(CH₂CH₂—O)ₓ—O—SO₃⁻M⁺, where R is a long-chained alkyl group, e.g. C₅-C₂₄-alkyl, x is 1-10 and M⁺ is a cation equivalent, generally Na⁺, K⁺, NH4⁺, mono-, di- or triethanolammonium); e.g. sodium laureth sulfate (SLES);
alkylbenzenesulfonates (generally of the formula R—(C₆H₄)—SO₃⁻M⁺, where R is a long-chained alkyl group, e.g. C₅-C₂₄-alkyl, and M⁺ is a cation equivalent, generally Na⁺, K⁺, NH₄⁺, mono-, di- or triethanolammonium); e.g. sodium dodecylbenzene sulfonate;
olefin sulfonates (generally of the formula R-SO₃⁻M⁺, where R is a long-chained monoolefin group, e.g. C₁₂-C₂₄-alkenyl, and M⁺ is a cation equivalent, generally Na⁺, K⁺, NH₄⁺, mono-, di-ortriethanolammonium); e.g. sodium C₁₄/C₁₆-a-olefin sulfonate;
alkane sulfonates (generally of the formula R-SO₃⁻M⁺, where R is a long-chained alkyl group, e.g. C₅-C₂₄-alkyl, and M⁺ is a cation equivalent, generally Na⁺, K⁺, NH₄⁺, mono-, di- or triethanolammonium); e.g. alkali metal or ammonium C₁₃-C₁₇ paraffin sulfonate;
sulfated monoglycerides (generally of the formula R—COO—CH₂—CH(OH)—CH₂—O—SO₃⁻M⁺, where R is a long-chained alkyl group, e.g. C₅-C₂₄-alkyl, and M⁺ is a cation equivalent, generally Na⁺, K⁺, NH₄⁺, mono-, di-ortriethanolammonium); e.g. sodium cocomonoglyceride sulfate;
alkyl sulfosuccinates, e.g. disodium N-octadecylsulfosuccinamate, diammonium lauryl sulfosuccinate, tetrasodium N-(1,2-dicarboxyethyl)-N-octadecylsulfosuccinate; diamyl ester of sodium sulfosuccinic acid, dihexyl ester of sodium sulfosuccinic acid, or dioctyl ester of sodium sulfosuccinic acid;
acyl taurates, e.g. N-alkyltaurins, e.g. prepared by reacting dodecylamine with sodiumisethionate or N-acyltaurines obtained by the reaction of N-methyl taurine with fatty acids;
acyl isethionates (generally of the formula R—COO—CH₂CH₂—SO₃⁻M⁺, where R is a long-chained alkyl group, e.g. C₁₀-C₃₀-alkyl, and M⁺ is a cation equivalent, generally Na⁺, K⁺, NH₄⁺, mono-, di- or triethanolammonium); e.g. ammonium cocoyl isethionate, sodium cocoyl isethionate or sodium lauroyl isethionate;
alkyl glycerylether sulfonates (generally of the formula R—O—CH₂—CH(OH)—CH₂—SO₃⁻M⁺, where R is a long-chained alkyl group, e.g. C₅-C₂₄-alkyl, and M⁺ is a cation equivalent, generally Na⁺, K⁺, NH₄⁺, mono-, di- or triethanolammonium); e.g. cocoglyceryl ether sulfonate;
sulfonated fatty acids and sulfonate fatty acid methyl esters (generally of the formula R—CH(SO₃M⁺)—COOH and R—CH(SO₃M⁺)—COOCH₃, where R is a long-chained alkyl group, e.g. C₅-C₂₄-alkyl, and M⁺ is a cation equivalent, generally Na⁺, K⁺, NH₄⁺, mono-, di- or triethanolammonium); e.g. a-sulfonated coconut fatty acid or lauryl methyl ester;
acyl glutamates (generally of the formula R—CO—N(COOH)—CH₂CH₂—COO⁻M⁺, where R is a long-chained alkyl group, e.g. C₅-C₂₄-alkyl, and M⁺ is a cation equivalent, generally Na⁺, K⁺, NH₄⁺, mono-, di- or triethanolammonium); e.g. sodium lauroyl glutamate or sodium cocoyl glutamate;
acyl sarcosinates (generally of the formula R—CO—N(CH₃)—CH₂—COO⁻M⁺, where R is a long-chained alkyl group, e.g. C₅-C₂₄-alkyl, and M⁺ is a cation equivalent, generally Na⁺, K⁺, NH₄⁺, mono-, di- or triethanolammonium); e.g. sodium lauroyl sarcosinate, sodium cocoyl sarcosinate or ammonium lauroyl sarcosinate;
alkyl sulfoacetates,
fatty acid salts, generally derived from the saponification of oils or fats, e.g. from palm oil or tallow oil, and having from 8 to 24 carbon atoms in the alkyl/alkenyl moiety (thus containing, inter alia, oleate, linolate, palmitate, myristate, stearate etc.), where the counter cation is generally Na⁺, K⁺, NH₄⁺, mono-, di- or triethanolammonium;
alkyl and alkenyl ether carboxylates (generally of the formula R—(OCH₂CH₂)ₓ—OCH₂—COO⁻M⁺, where R is a long-chained alkyl or alkenyl group, e.g. C₈-C₂₄-alkyl or -alkenyl, x is 1 to 10 and M⁺ is a cation equivalent, generally Na⁺, K⁺, NH₄⁺, mono-, di- or triethanolammonium); e.g. sodium laureth carboxylate;
acylated peptides,
acyl lactylates (generally of the formula R—CO—[OCH(CH₃)—CO]ₓ—COO⁻M⁺, where R is a long-chained alkyl or alkenyl group, e.g. C₅-C₂₄-alkyl or -alkenyl, x is 3 and M⁺ is a cation equivalent, generally Na⁺, K⁺, NH₄⁺, mono-, di- or triethanolammonium); e.g. sodium cocoyl lactylate.

Another class of suitable additional anionic surfactants are polyalkoxylate polycarboxylated surfactants, e.g. as described in U.S. Pat. No. 5,376,298, EP-A-0129328, WO 03/018733 U.S. Pat. No. 5,120,326. The polyalkoxylate polycarboxylated surfactant can be described by the formula R—O—(C₂H₄O)ₓ—[CH(L)CH(L)]_{y}-[CH₂CH(CH₃)O)_{z}Q, wherein R is a hydrophobic hydrocarbon group, preferably alkyl, containing from 6 to 16, preferably from 8 to 14 carbon atoms; x is a number from 0 to 60, preferably from 4 to 50, more preferably from 6 to 50; L is either a C₁-C₃ alkyl group or a group having the formula —CH(COO⁻)—CH₂(COO⁻), with at least one L group in each molecule being —CH(COO⁻)—CH₂(COO⁻); y is a number from 1 to 12, preferably from 2 to 10, more preferably from 3 to 8; z is a number from 0 to 20, preferably from 0 to 15, more preferably from 0 to 10; and Q is selected from the group consisting of H and sulfonate groups, the compound being rendered electrically neutral by the presence of cationic groups, preferably selected from the group consisting of sodium, potassium, and substituted ammonium, e.g. monoethanol ammonium, cations. Such polyalkoxylate polycarboxylate surfactants are commercially available under the Plurafac® brand of BASF, e.g. Plurafac® CS-10.

Cationic surfactants are, for example, ammonium salts such as C₈-C₁₆-dialkyldimethylammonium halides, dialkoxydimethylammonium halides or imidazolinium salts with a long-chain alkyl radical.

Non-ionic surfactants are typically the condensation products of one or more alkylene oxide, mostly ethylene oxide, with various reactive hydrogen-containing compounds having hydrophobic chains, for example with 8-24 carbon atoms, e.g. the condensation products of polyethyleneoxide with fatty alcohols, long chain branched alkyl alcohols, fatty acids, fatty amines, polyhydric alcohols or polypropylene oxide.

Suitable alkoxylated alcohols are listed in the following. They are of course only suitable as component (c) if they differ from the alkoxylated alkanols of component (b), i.e. if they are no C₆-C₁₄-alkanols alkoxylated with 3 to 5 units of ethylene oxide on average, e.g. if they contain more than 5 or less than 3 units of ethylene oxide on average, or alkoxylated with a total of 3 to 5 units of ethylene oxide and propylene oxide on average; if they have less than 6 or more than 14 carbon atoms in the alcohol part, if the alcohol part is not derived from an alkanol and/or if they are alternatively or additionally alkoxylated with another alkylene oxide (e.g. only with propylene oxide, PO) or alkylene diol (e.g. only with propylene-1,2- or with 1,3-diol).

Suitable alkoxylated, advantageously ethoxylated, alcohols are especially alkoxylated, advantageously ethoxylated, primary alcohols having preferably 8 to 18 carbon atoms and an average of 1 to 20, preferably 1 to 12, mol of ethylene oxide (EO) per mole of alcohol, in which the alcohol radical may be linear or branched, in particular 2-methyl-branched, or may comprise linear and methyl-branched radicals in a mixture, as are typically present in oxo alcohol radicals. Also suitable are alkyl alcohols synthesized by the Guerbet process, for example, 2-ethylhexanol, 2-n-propylheptanol, 2-isopropyl-heptanol, 2-n-butyloctanol, and 2-n-pentylnonanol, preferred are 2-ethylhexanol, 2-n-propylheptanol, and 2-isopropyl-heptanol. More preference is given to 2-n-propylheptanol. Nonionic surfactants synthesized from this latter alcohol are marketed by BASF under the brand names Lutensol® XP and Lutensol® XL.

Other preferred ethoxylated alkyl alcohols have a higher degree of branching, especially ethoxylated alkyl alcohols available under the BASF brand names Lutensol® TO Lutensol® ON and Lutensol® TDA.

Suitable are also alcohol ethoxylates with linear radicals formed from alcohols of native origin having 12 to 18 carbon atoms, for example from coconut alcohol, palm alcohol, tallow fat alcohol or oleyl alcohol, and an average of 2 to 12 EO per mole of alcohol.

The preferred ethoxylated alcohols include, for example, C₁₂-C₁₄-alcohols with 7 EO or 10 EO, C₉-C₁₁-alcohol with 7 EO or 10 EO, C₁₃-C₁₅-alcohols with 7 EO, 8 EO or 10 EO, C₁₂-C₁₈-alcohols with 7 EO or 10 EO and mixtures thereof. The degrees of ethoxylation stated are statistical averages which, for a specific product, may be an integer or a fraction. Also suitable are alcohol ethoxylates which have a narrowed homolog distribution (narrow range ethoxylates, NRE). In addition to these alkoxylated alcohols, it is also possible to use fatty alcohols with more than 12 EO. Examples thereof are tallow fat alcohol with 14 EO, 25 EO or 30 EO. It is also possible to use alkoxylated alcohols which comprise EO and PO groups together in the molecule. In this case, it is possible to use block copolymers with EO-PO block units or PO-EO block units, but also EO-PO-EO copolymers or PO-EO-PO copolymers. It will be appreciated that it is also possible to use mixed-alkoxylation nonionic surfactants in which EO and PO units are not in blockwise but in random distribution. Such products are obtainable by the simultaneous action of ethylene oxide and propylene oxide on fatty alcohols.

Suitable alkoxylated, preferably ethoxylated or ethoxylated and propoxylated, fatty acid alkyl esters preferably have 1 to 4 carbon atoms in the alkyl chain and are especially fatty acid methyl esters.

Non-ethoxylated non-ionic surfactants are for example sugar surfactants, glycerol monoethers, polyhydroxyamides (glucamide) or amine oxides.

Sugar surfactants are for example alkyl and/or alkenyl polyglycosides, sugar or alkyl sugar fatty acid esters, and fatty sugar amides.

Alkyl and/or alkenyl polyglycosides are nonionic surfactants with a carbohydrate as hydrophilic moiety and fatty alcohols or fatty acids as hydrophobic component. Examples are compounds of the formula

R—O-Gₚ,

where R is a long-chained alkyl or alkenyl group, mostly with 4-22 carbon atoms, G is an aldose or ketose moiety, mostly a glucose moiety, and p is from 1 to 10.

G is preferably derived from aldoses or ketoses having 5 or 6 carbon atoms. In one embodiment, component G is selected from the group of hexoses, preferably from the group consisting of allose, altrose, glucose, mannose, gulose, idose, galactose, talose, psicose, fructose, sorbose and tagatose, and is more preferably glucose. In another embodiment, component G is selected from the group of pentoses, preferably from the group consisting of ribulose, xylulose, ribose, arabinose, xylose and lyxose, and more preferably from xylose and arabinose.

The index number p in the above formula gives the degree of polymerization (DP), and is a number between 1 and 10. In one embodiment p is of from 1.1 to 3.0.

R can be linear or branched. For instance, the radical R is derived from linear primary alcohols, e.g. fatty alcohols, or from branched primary alcohols, in particular so-called oxo alcohols. Examples for R derived from linear primary alcohols are n-octyl, n-nonyl, n-decyl, n-undecyl, n-dodecyl, n-tridecyl, n-tetradecyl, n-pentadecly, n-heptadecyl or n-octadecyl. Examples for R derived from branched primary alcohols are isoamyl, isohexyl, isoheptyl, 2-ethylhexyl and 2-propylheptyl.

It is also possible to use mixtures of different alkyl and/or alkenyl polyglycosides Thus, all combinations of the various aldoses or ketoses with all possible alkyl- and/or alkenyl radicals can be used.

Commercially available alkyl and/or alkenyl polyglycosides are for example products sold under the PLANATAREN® and PLANTACARE® brands from Henkel, e.g. PLANTAREN 1200, PLANTAREN 1300, PLANTAREN 2000, PLANTACARE 2000, PLANTACARE 818, PLANTACARE 1200; products sold under the TRITON® CG brand from Seppic, e.g. TRITON CG 110 (or ORAMIX CG 110) and TRITON CG 312 (or ORAMIX NS 10); the product sold as LUTENSOL® GD 70 from BASF SE; the products sold under the Glucopon® brand from BASF SE, e.g. Glucopon 100 DK, Glucopon215 UP, Glucopon 225 DK, Glucopon 425 N/HH, Glucopon GD 70, Glucopon 50 G, Glucopon 600 CSUP or Glucopon 650 EC; and the product Plantatex® LLE from BASF SE.

Sugar or alkyl sugar fatty acid esters are sugar or alkyl sugar C₄-C₂₂ fatty acid esters among which there may be mentioned in particular:

(C₁-C₄)alkyl glucoside esters such as methyl glucoside monostearate, e.g. the product sold under the name GRILLOCOSE® IS by Grillowerke; methyl glucoside sesquistearate, e.g. the product sold under the name GLUCATE SS by Amerchol; 6-ethylglucoside decanoate, e.g. the product sold under the name BIOSURF 10 by Novo; the mixture of mono- and dicocoate (82/7) of 6-ethylglucoside, e.g. the product sold under the name BIOSURF® COCO by Novo; the mixture of mono- and dilaurate (84/8) of 6-ethylglucoside, e.g. the product sold under the name BIOSURF® 12 by Novo; the butyl glucoside C₁₂-C₁₈ fatty acid monoesters, such as butyl glucoside monococoate, e.g. the product sold under the names REWOPOL® V3101 or REWOSAN® V3101 and polyoxyethylenated butyl glucoside monococoate with 3 moles of ethylene oxide, e.g. the product sold under the name REWOPOL® V3122 by Rewo; glucose esters, such as 6-O-hexadecanoyl-[alpha]-D-glucose, 6-O-octanoyl-D-glucose, 6-O-oleyl-D-glucose, 6-O-linoleyl-D-glucose, which can be prepared, for example, from the corresponding acid chloride and D-glucose; sucrose monoesters such as sucrose monolaurate, e.g. the product sold under the name GRILLOTEN® LES 65, and sucrose monococoate sold under the name GRILLOTEN® LES 65K sold by the company Grillo-Werke.

The fatty sugar amides are compounds comprising at least one amide function and including at least one sugar or sugar derivative portion and at least one fatty chain; such compounds may, for example, result from the action of a fatty acid or a fatty acid derivative on the amine function of an amino sugar, or from the action of a fatty amine on a sugar comprising a carboxylic acid function (free or in lactone form) or carboxylic acid-derived function or alternatively a carbonyl function, and optionally in the presence of suitable co-reagents. Examples are N-substituted aldonamides polyhydroxylated fatty acid amides or mixtures thereof.

The N-substituted aldonamides are for example N-substituted lactobionamides, N-substituted maltobionamides, N-substituted cellobionamides, N-substituted mellibionamides and N-substituted gentiobionamides such as:

N-alkyllactobionamides, N-alkylmaltobionamides, N-alkylcellobionamides, N-alkylmellibionamides or N-alkylgentiobionamides which are mono- or disubstituted with a saturated or unsaturated, linear or branched, aliphatic hydrocarbon group which may contain heteroatoms preferably having up to 36 carbon atoms, more preferably up to 24 carbon atoms and still more particularly from 8 to 18 (for example methyl, ethyl, amyl, hexyl, heptyl, nonyl, decyl, undecyl, dodecyl, tridecyl, tetradecyl, pentadecyl, hexadecyl, heptadecyl, octadecyl; allyl, undecenyl, oleyl, linoleyl, propenyl, heptenyl), with an aromatic hydrocarbon group (for example benzyl, aniline, substituted benzyl, phenylethyl, phenoxyethyl, vinylbenzyl) or cycloaliphatic groups (for example cyclopentyl, cyclohexyl);

N-lactobionylamino acid esters where the amino acid may denote in particular: alanine, valine, glycine, lysine, leucine, arginine, aspartic acid, glutamic acid, threonine, serine, cysteine, histidine, tyrosine, methionine or which may be chosen, for example, from [beta]-alanine, sarcosine, gamma-aminobutyric acid, ornithine, citrulline or their equivalents; the said N-lactobionylamino acid esters being monosubstituted with a group of formula —(CH₂)ₙ—C(═O)—OR, where R is an aliphatic hydrocarbon group which may contain up to 36 carbon atoms and n is an integer greater than 1, as well as the corresponding N-maltobionylamino acid esters, the N-mellibionylamino acid esters, the N-cellobionylamino acid esters and the N-gentiobionylamino acid esters;

N-(alkyloxy)alkyllactobionamides which are mono- or disubstituted with a group —(CH₂)ₙ—OR′, where R′ is an aliphatic, aromatic or cycloaliphatic hydrocarbon group; N-(polyalkyloxy)alkyllactobionamides, N-(polyalkyloxy)alkylmaltobionamides, N-(polyalkyloxy)alkylcellobionamides, N-(polyalkyloxy)alkylmellibionamides or N-(polyalkyloxy)alkylgentiobionamides which are mono- or disubstituted with a group —R′—(OR′)ₙR′R″ where R′ is an alkylene group such as ethylene, propylene or mixtures thereof, n is an integer greater than 1, R″ is a lactobionamide, maltobionamide, cellobionamide, mellibionamide or gentiobionamide group.

Examples for polyhydroxylated fatty amides are compounds of the formula T-C(═O)—N(V)—W where T denotes a C₅-C₃₁ hydrocarbon group, preferably a C₇-C₁₅ linear alkyl or alkenyl chain; V denotes hydrogen, a C₁-C₄ hydrocarbon radical, 2-hydroxyethyl, 2-hydroxypropyl or mixtures thereof, preferably a C₁-C₄ alkyl such as methyl, ethyl, propyl, isopropyl, N-butyl and more particularly methyl; and W denotes a polyhydroxy hydrocarbon-containing group having a linear hydrocarbon chain with at least 3 hydroxyl groups directly attached to the chain, or an alkoxylated derivative of the said group (preferably ethoxylated or propoxylated).

W is preferably a reducing sugar derivative obtained by reductive amination reaction and more preferably a glycityl group. Glucose, maltose, lactose, galactose, mannose and xylose may be mentioned among the reducing sugars. Preferably, W is chosen from the groups of the following formulae: —(CH₂)—(CHOH)ₙ—CH₂OH; —CH—(CH₂OH)—(CHOH)ₙ₋₁—CH₂OH; and —CH₂—(CHOH)₂(CHOR′)—(CHOH)—CH₂OH, in which n is an integer ranging from 3 to 5, and R′ is hydrogen, a cyclic or aliphatic monosaccharide or one of its alkoxylated derivatives. A glycityl group in which n is 4, and in particular the group —(CH₂)—(CHOH)₄—CH₂OH, is preferred.

The group T-C(═O)—N— may be for example cocamide, stearamide, oleamide, lauramide, myristiramide, capricamide, palmitamide, tallow amide.

Non-ionic surfactants of the amine oxide type are generally of the formula R^{a}R^{b}R^{c}N⁺—O⁻, where R^{a} is a long-chained alkyl group, e.g. C₁₀-C₁₈-alkyl, preferably C₁₂-C₁₆-alkyl, and R^{b} and R^{c} are short-chained alkyl or hydroxyalkyl groups, such as methyl, ethyl or 2-hydroxyethyl. A specific example is lauryldimethylamine oxide. Moreover, the long-chained alkyl group can be derived from native sources (oils or fats), resulting in mixtures of such amine oxides, for example N-cocoalkyl-N,N-dimethylamine oxide and N-tallowalkyl-N,N-dihydroxyethylamine oxide.

Amphoteric surfactants are, for example, derivatives of secondary or tertiary amines, for example C₆-C₁₈-alkyl betaines (e.g. cocoamidopropyl betaine; disodium cocoamphodiacetate (DSCADA)) or C₆-C₁₈-alkyl sulfobetaines, or amine oxides such as alkyldimethylamine oxides.

C₂-C₃-Alkanols [component (d)] are ethanol, n-propanol and isopropanol. Mixtures thereof are also suitable.

The organic solvents different from component (d) [component (e)] generally serve for providing a stable cleaning composition, especially if the cleaning composition is a concentrate containing high amounts of organic matter.

Suitable solvents are thus polar protic or polar aprotic. Examples for suitable solvents (e) are alkanols different from C₂-C₃-alkanols, such as n-butanol or tert-butanol; C₂-C₈-alkanediols; C₁-C₈-alkylmonoethers of C₂-C₈-alkanediols; diglycols, C₁-C₈-alkylmonoethers of diglycols, polyetherpolyols; C₁-C₈-alkylmonoethers of polyetherpolyols; amino alcohols, such as ethanolamine, diethanolamine and triethanolamine; monophenyl ethers of C₂-C₃-alkanediols, such as 2-phenoxyethanol or phenoxypropanol.

Among the above solvents, preference is given to C₂-C₈-alkanediols and C₁-C₈-alkylmonoethers of C₂-C₈-alkanediols and to C₁-C₈-alkylmonoethers of diglycols. More preference is given to C₂-C₄-alkanediols, in particular ethylene glycol and propylene glycol, C₁-C₄-alkylmonoethers of a C₂-C₃-alkanediol, such as the C₁-C₄-alkylmonoethers of ethylene glycol or propylene glycol, specific examples being ethylene glycol monomethyl ether, ethylene glycol monoethyl ether, ethylene glycol mono-n-propyl ether, ethylene glycol mono-n-butyl ether (also termed butylglyol), propylene glycol monomethyl ether, propylene glycol monoethyl ether, propylene glycol mono-n-propyl ether, and propylene glycol mono-n-butyl ether; and to C₁-C₄-alkylmonoethers of diglycols, for example butyldiglycol, and n-hexanol ethoxylated with 1-3EO and mixtures thereof.

Sequestrants [components (f)], also termed builders, structural substances, framework substances, complexing agents, chelators, chelating agents or softeners, bind alkaline earth metals and other water-soluble metal salts without precipitating. They help to break up soil, disperse soil components, help to detach soil and in some cases themselves have a washing effect. Many of the sequestrants listed below are multi-functional, meaning that the substances have additional functions, such as a dispersing activity.

Suitable sequestrants may be either organic or inorganic in nature. Examples are aluminosilicates, carbonates, phosphates and polyphosphates, polycarboxylic acids, polycarboxylates, hydroxycarboxylic acids, phosphonic acids, e.g. hydroxyalkylphosphonic acids, phosphonates, aminopolycarboxylic acids and salts thereof, and polymeric compounds containing carboxylic acid groups and salts thereof.

Suitable inorganic sequestrants are, for example, crystalline or amorphous aluminosilicates with ion-exchanging properties, such as zeolites. Crystalline silicates suitable as sequestrants are, for example, disilicates or sheet silicates, e.g. d-Na₂Si₂O₅ or b-Na₂Si₂O₅ (SKS 6 or SKS 7). Suitable inorganic sequestrant substances based on carbonate are carbonates and hydrogencarbonates. These can be used in the form of their alkali metal, alkaline earth metal or ammonium salts. Customary phosphates used as inorganic sequestrants are alkali metal orthophosphates and/or polyphosphates, for example pentasodium triphosphate.

Suitable organic sequestrants are, for example, C₄-C₃₀-di-, -tri- and -tetracarboxylic acids, for example succinic acid, propanetricarboxylic acid, butanetetracarboxylic acid, cyclopentanetetracarboxylic acid, and alkyl- and alkenylsuccinic acids with C₂-C₂₀-alkyl or -alkenyl radicals. Suitable organic sequestrants are also hydroxycarboxylic acids and polyhydroxycarboxylic acids (sugar acids). These include C₄-C₂₀-hydroxycarboxylic acids, for example malic acid, tartaric acid, glutonic acid, mucic acid, lactic acid, glutaric acid, citric acid, tartronic acid, glucoheptonic acid, lactobionic acid, and sucrosemono-, -di- and -tricarboxylic acid. Among these, preference is given to citric acid and salts thereof. Another class are carboxylated fructans. Fructans are polymers of fructose molecules. They are built up of fructose residues, normally with a sucrose unit (i.e. a glucose-fructose disaccharide) at what would otherwise be the reducing terminus. The linkage of the fructose residues normally occurs at one of the two primary hydroxyls (OH-1 or OH-6). In inulin, the fructosyl residues are linked by b-2,1-linkages. In levan and phlein, the fructosyl residues are linked by b-2,6-linkages. The graminin type contains both b-2,1-linkages and b-2,6-linkages. Preferably, the carboxylated fructans are derived from inulin. Particular examples are carboxymethylinulin and carboxyethylinulin. Suitable carboxylated fructans are described in EP 3561032 A1 and WO 2010/106077.

Suitable organic sequestrants are also phosphonic acids, for example hydroxyalkylphosphonic acids or aminophosphonic acids, and the salts thereof. These include, for example, phosphonobutanetricarboxylic acid (2-phosphinobutane-1,2,4-tricarboxylic acid; PBTC), aminotris-methylenephosphonic acid (N[CH₂PO(OH)₂]₃), aminotris(methylenephosphonate), sodium salt (ATMP; N[CH₂PO(ONa)₂]₃), ethylenediaminetetra(methylenephosphonic acid) (EDTMPA), hexamethylenediamine(tetramethylenephosphonic acid), hexamethylenediamine(tetramethylenephosphonate), potassium salt (C₁₀H₍₂₈₋ₓ₎N₂KₓO₁₂P₄ (x=6)), bis(hexamethylene)triamine(pentamethylenephosphonic acid) ((HO₂)POCH₂N[(CH₂)₂N[CH₂PO(OH)₂]₂]₂), diethylenetriamine-penta(methylene-phosphonic acid) (DTPMP; (HO)₂POCH₂N[CH₂CH₂N[CH₂PO(OH)₂]₂]₂), diethylenetriaminepenta(methylenephosphonate), sodium salt (C₉H₍₂₈₋ₓ₎N₃NaₓO₁₅P₅ (x=7)); tetramethylene-triamine-pentaphosphonic acid, hydroxyethylamine diphosphonic acid, 2-hydroxyethyliminobis(methylenephosphonic acid) (HOCH₂CH₂N[CH₂PO(OH)₂]₂), morpholinomethanediphosphonic acid, 1-hydroxy-C₁- to C₁₀-alkyl-1,1-diphosphonic acids such as 1-hydroxyethane-1,1-diphosphonic acid (HEDP; CH₂C(OH)[PO(OH)₂]₂). Suitable organic sequestrants are moreover polyasparatic acids. Polyaspartic acid include salts of polyaspartic acids. Salt forming cations may be monovalent or multivalent, examples being sodium, potassium, magnesium, calcium, ammonium, and the ammonium salt of mono-, di- and triethanolamine. Such polymers may be co-polymers, in particular of (a) L- or D-aspartic acid (preferably L-aspartic acid), (b) a carboxylic acid and (c) a diamone or an amino alcohol. Such copolymers generally comprise 70-95 mol % of (a), 5-30 mol % of (b) and 2-20 mol % of (c). The molar ratio of the carboxyl-containing compound (b) to the diamine or amino alcohol (c) is preferably between 5:1 and 1:1.5 or between 3:1 and 1:1.2, and more preferably between 3:1 and 1:1 or 2:1 and 1:1. Suitable organic sequestrants are additionally aminopolycarboxylic acids, such as nitrilotriacetic acid (NTA), nitrilomonoacetic dipropionic acid, nitrilotripropionic acid, b-alaninediacetic acid (b-ADA), ethylenediaminetetraacetic acid (EDTA), diethylenetriaminepentaacetic acid (DTPA), 1,3-propylenediaminetetraacetic acid, 1,2-propylenediaminetetraacetic acid, N-(alkyl)ethylenediaminetriacetic acid, N-(hydroxyalkyl)ethylenediaminetriacetic acid, ethylenediaminetriacetic acid, cyclohexylene-1,2-diaminetetraacetic acid, iminodisuccinic acid, ethylenediaminedisuccinic acid, serinediacetic acid, isoserinediacetic acid, L-asparaginediacetic acid, L-glutaminediacetic acid, methylglycinediacetic acid (MGDA), and the salts of the aforementioned aminopolycarboxylic acids. Suitable organic sequestrants are additionally polymeric compounds containing carboxylic acid groups, such as acrylic acid homopolymers. The term “acrylic acid homopolymer” also comprises polymers in which some or all of the carboxylic acid groups are present in neutralized form. Suitable polymeric compounds containing carboxylic acid groups are also oligomaleic acids. Suitable polymeric compounds containing carboxylic acid groups are also terpolymers of unsaturated C₄-C₈-dicarboxylic acids. Suitable unsaturated C₄-C₈-dicarboxylic acids in this context are, for example, maleic acid (or maleic anhydride), fumaric acid, itaconic acid, aconitic acid, mesaconic acid, methylenemalonic acid and citraconic acid. Suitable polymeric compounds containing carboxylic acid groups are also homopolymers of the monoethylenically unsaturated C₃-C₈-monocarboxylic acids, for example acrylic acid, methacrylic acid, crotonic acid, 2-ethylacrylic acid, 2-phenylacrylic acid, cinnamic acid, vinylacetic acid and sorbic acid, copolymers of dicarboxylic acids, for example of maleic acid and acrylic acid; terpolymers of maleic acid, acrylic acid and a vinyl ester of a C₁-C₃-carboxylic acid; and copolymers of maleic acid with C₂-C₈-olefins.

Further additives [component (g)] are for example pH adjusting agents (pH modifiers), thickeners, antifreezing agents, antifoaming agents, colorants and perfumes [i.e. different from components (a) and (b)].

Depending on the desired pH of the cleaning composition, pH adjusting agents (pH modifiers) are acids or bases. The pH can also be adjusted by buffering systems.

The acids can be inorganic or organic. Suitable inorganic acids are for example sulfuric acid, hydrochloric acid and phosphoric acid, where sulfuric acid is generally preferred. Suitable organic acids are for example aliphatic, saturated non-substituted C₁-C₆-mono-, di- and tri-carboxylic acids such as formic acid, acetic acid, propanoic acid, oxalic acid, succinic acid, glutaric acid and adipic acid; aliphatic, saturated C₁-C₆-mono-, di- and tri-carboxylic acids carrying one or more OH groups, such as glycolic acid, lactic acid, tartric acid and citric acid; aliphatic, unsaturated C₁-C₆-mono-, di- and tri-carboxylic acids such as sorbic acid; aromatic carboxylic acids, such as benzoic acid, salicylic acid and mandelic acid; and sulfonic acids, such as methanesulfonic acid or toluenesulfonic acid. The organic acids mainly serve for adapting the pH of the cleaning composition, but some of them, e.g. the di- and tricarboxylic acids, can also act as sequestrants.

Suitable bases are in particular inorganic bases, such as the carbonates mentioned in context with the sequestrant, e.g. sodium or potassium carbonate; further ammonium carbonate, alkali metal and earth alkaline metal bicarbonates, such as sodium hydrogencarbonate or potassium hydrogencarbonate, alkali metal and earth alkaline metal hydroxides, such as NaOH or KOH, or ammonium hydroxide. Organic bases can also be used; examples are alkanolamines, such as monoethanolamine, triethanolamine or aminomethylpropanol, or guanidine derivatives, such as 1,1,3,3-tetramethylguanidine or triazabicyclodecene.

Suitable buffering agents are the typical systems, such as hydrogenphosphate/dihydrogenphosphate buffer, carbonate/hydrogencarbonate buffer, acetic acid/acetate buffer or Tris buffer. Moreover, most of the above acids which are weak and the anion of which is not a strong salt also have buffering capacity.

The thickeners serve to impart the desired viscosity to the cleaning composition of the invention.

Any known thickener (rheology modifier) is suitable in principle, provided that it does not exert any adverse effect on the efficacy of the cleaning composition. Suitable thickeners may either be of natural origin or of synthetic nature.

Thickeners of natural origin are mostly derived from polysaccharides. Examples are xanthan, gellan gum, carob flour, guar flour or gum, carrageenan, agar, tragacanth, gum arabic, alginates, modified starches such as hydroxyethyl starch, starch phosphate esters or starch acetates, dextrins, pectins and cellulose derivatives, such as carboxymethylcellulose, hydroxyethylcellulose, hydrophobically modified hydroxyethyl cellulose, hydroxypropylcellulose, hydroxypropylmethylcellulose, methylcellulose and the like. Thickeners of natural origin are also inorganic thickeners, such as polysilicic acids and clay minerals, for example sheet silicates, and also the silicates mentioned for the builders.

Examples of synthetic thickeners are polyacrylic and polymethacrylic compounds, such as (partly) crosslinked homopolymers of acrylic acid, for example homopolymers of acrylic acid which have been crosslinked with an allyl ether of sucrose or pentaery-thritol, or with propylene (carbomers), for example the Carbopol® brands from BF Goodrich (e.g. Carbopol® 676, 940, 941, 934 and the like) or the Polygel® brands from 3V Sigma (e.g. Polygel® DA), copolymers of ethylenically unsaturated mono- or dicarboxylic acids, for example terpolymers of acrylic acid, methacrylic acid or maleic acid with methyl acrylate or ethyl acrylate and a (meth)acrylate which derives from long-chain ethoxylated alcohols, for example the Acusol® brands from Rohm & Haas (e.g. Acusol® 820 or 1206A), copolymers of two or more monomers which are selected from acrylic acid, methacrylic acid and the C₁-C₄-alkyl esters thereof, for example copolymers of methacrylic acid, butyl acrylate and methyl methacrylate or of butyl acrylate and methyl methacrylate, for example the Aculyn® and Acusol® brands from Rohm & Haas (e.g. Aculyn® 22, 28 or 33 and Acusol® 810, 823 and 830), or crosslinked high molecular weight acrylic acid copolymers, for example copolymers of C₁₀-C₃₀-alkyl acrylates with one or more comonomers selected from acrylic acid, methacrylic acid and the C₁-C₄-alkyl esters thereof, said copolymers having been crosslinked with an allyl ether of sucrose or pentaerythritol (e.g. Carbopol® ETD 2623, Carbopol® 1382 or Carbopol® AQUA 30 from Rohm & Haas). Another preferred substance group is the Rheovis® brands from BASF, e.g. Rheovis® AT 120.

Examples for suitable antifreezing agents are ethylene glycol, propylene glycol, urea and glycerine.

Examples for suitable antifoaming agents are silicones, long-chain alcohols and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Fragrances can be of natural or synthetic origin, their nature is in general not critical.

Moreover, the cleaning composition may comprise enzymes [component (h)]. Preferably, the at least one enzyme is a detergent enzyme.

In one embodiment, the enzyme is classified as an oxidoreductase (EC 1), a transferase (EC 2), a hydrolase (EC 3), a lyase (EC 4), an isomerase (EC 5), or a ligase (EC 6). The EC-numbering is according to Enzyme Nomenclature, Recommendations (1992) of the Nomenclature Committee of the International Union of Biochemistry and Molecular Biology including its supplements published 1993-1999. Preferably, the enzyme is a hydrolase (EC 3).

In a preferred embodiment, the enzyme is selected from the group consisting of proteases, amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, beta-glucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the enzyme is selected from the group consisting of proteases, amylases, lipases, cellulases, mannanases, xylanases, DNases, dispersins, pectinases, oxidoreductases, and cutinases, and combinations of at least two of the foregoing types. Most preferably, the enzyme is a protease, preferably, a serine protease, more preferably, a subtilisin protease.

Preferably, the protease is a protease with at least 90% sequence identity to SEQ ID NO: 22 of EP1921147B1 and having the amino acid substitution R101E (according to BPN′ numbering). Preferably, the amylase is an amylase with at least 90% sequence identity to SEQ ID NO: 54 of WO2021032881A1.

The composition of the present invention can comprise one type of enzyme or more than one enzyme of different types, e.g., an amylase and a protease, or more than one enzyme of the same type, e.g., two or more different proteases, or mixtures thereof, e.g., an amylase and two different proteases.

The enzyme(s) can be incorporated into the composition at levels sufficient to provide an effective amount for achieving a beneficial effect, preferably for primary washing effects and/or secondary washing effects, like anti-greying or antipilling effects (e.g., in case of cellulases). Preferably, the enzyme is present in the composition at levels from about 0.00001% to about 5%, preferably from about 0.00001% to about 2%, more preferably from about 0.0001% to about 1%, or even more preferably from about 0.001% to about 0.5% enzyme protein by weight of the composition.

Preferably, the enzyme-containing composition further comprises an enzyme stabilizing system.

Preferably, the enzyme-containing composition described herein comprises from about 0.001% to about 10%, from about 0.005% to about 8%, or from about 0.01% to about 6%, by weight of the composition, of an enzyme stabilizing system. The enzyme stabilizing system can be any stabilizing system which is compatible with the enzyme.

Preferably, the enzyme stabilizing system comprises at least one compound selected from the group consisting of polyols (preferably, 1,3-propanediol, ethylene glycol, glycerol, 1,2-propanediol, or sorbitol), inorganic salts (preferably, CaCl2, MgCl2, or NaCl), short chain (preferably, C1-C3) carboxylic acids or salts thereof (preferably, formic acid, formate (preferably, sodium formate), acetic acid, acetate, or lactate), borate, boric acid, boronic acids (preferably, 4-formyl phenylboronic acid (4-FPBA)), peptide aldehydes (preferably, Z-VAL-H or Z-GAY-H), peptide acetals, and peptide aldehyde hydrosulfite adducts. Preferably, the enzyme stabilizing system comprises a combination of at least two of the compounds selected from the group consisting of salts, polyols, and short chain carboxylic acids and preferably one or more of the compounds selected from the group consisting of borate, boric acid, boronic acids (preferably, 4-formyl phenylboronic acid (4-FPBA)), peptide aldehydes, peptide acetals, and peptide aldehyde hydrosulfite adducts. In particular, if proteases are present in the composition, protease inhibitors may be added, preferably selected from borate, boric acid, boronic acids (preferably, 4-FPBA), peptide aldehydes (preferably, peptide aldehydes like Z-VAL-H or Z-GAY-H), peptide acetals, and peptide aldehyde hydrosulfite adducts.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all these publications and those references cited within those publications in their entireties are hereby incorporated by reference into this application in order to more fully describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of members, this is meant to also encompass a group which consists of these members only.

The present invention is further illustrated by the following examples, sequences and FIGURES from which further features, embodiments, aspects and advantages of the present invention may be taken. Further, the materials, methods, and examples are illustrative only and are not intended to be limiting, unless otherwise specified.

### DETAILED DESCRIPTION OF THE FIGURES

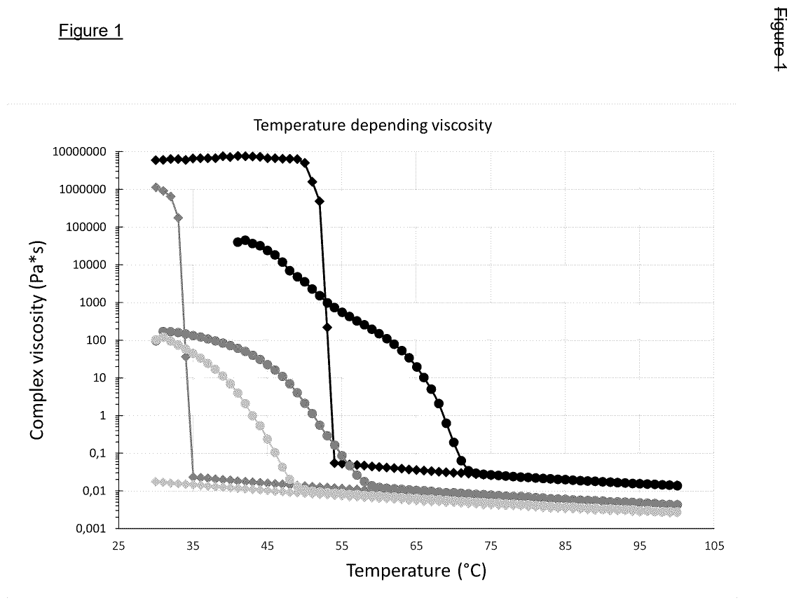

FIG. 1 shows the temperature depending viscosities of pure C12/C14-isosorbid monoester (black graph) in comparison to the ones from a 50:50 (grey) and a 30:70 (bright grey) mixture of C12/C14-isosorbid monoester with C12/C14-fatty alcohol. Dotted lines show the heating from 30/40 °C to 100 °C and diamond lines the cooling from 100 °C to 25/30 °C.

### EXAMPLES

### A) Synthesis of Isosorbide Ester Sulfate

### i) Synthesis of Isosorbide Monoester

### Example 1

In a 2 L flask with 2-blade stirrer, heating mantle and condenser, to 8 mol Isosorbide (1169.6 g; 146.2 g/mol), 1 mol Myristic Acid (230.0 g; 228.4 g/mol; C14) and 1 mol Lauric Acid (202.0 g; 200.3 g/mol; C12) were added.

The substances were heated to 100 °C under light nitrogen purge. At 110 °C (substances completely liquid, 2 phases), the stirrer was activated (250 rpm) and the temperature was raised to 160 °C. At this temperature the catalyst was added (Tin (II) oxalate 0.5 g) and the temperature was raised to 225 °C at a rate of 25°/h.

Condensation started at 185 °C. The reaction temperature was held for 4 h till the end of condensation. The temperature of 225 °C was held for another 1.5 h. Then the mixture was cooled to 130 °C, at this temperature 10 g water were added to disactivate the catalyst. The temperature of 130 °C was held for another 30 minutes. Subsequently the excess of Isosorbide was stripped by distillation at 225 °C/10 mbar, the remaining raw product was cooled to 100 °C.

Reaction control was achieved by gas chromatography. The process yielded 535.5 g dark brownish, waxy product, 85% of theorical yield (686.4 g).

### ii) Sulfation

### Example 2

In a continuously operating falling film reactor with jacket cooling and overhead SO₃ fumigation, 600 g mixture of 30% C12/14 isosorbide ester and 70% C12/14 fatty alcohol (Lorol® series as commercially available from BASF) were reacted with 259.7 g SO₃. The SO₃ was expelled by heating from 399.8 g of 65% oleum, diluted with nitrogen to 5% by volume and brought into contact with the isosorbide-fatty alcohol mixture film via a nozzle. The crude sulphation product was neutralized in water containing 1% sodium dihydrogen phosphate×1H₂O, and the pH was continuously adjusted to 6.5-7.5 with 50% sodium hydroxide solution under cooling to <40 °C and mixing with an ultra-turrax.

### Example 3

In a continuously operating falling film reactor with jacket cooling and overhead SO₃ fumigation, 600 g mixture of 50% C12/14 isosorbide ester and 50% C12/14 fatty alcohol (Lorol® series as commercially available from BASF) were reacted with 234.8 g SO₃.

The SO₃ was expelled by heating from 361.5 g of 65% oleum, diluted with nitrogen to 5% by volume and brought into contact with the isosorbide-fatty alcohol mixture film via a nozzle. The crude sulphation product was neutralized in water containing 1% sodium dihydrogen phosphate×1H₂O, and the pH was continuously adjusted to 6.5-7.5 with 50% sodium hydroxide solution under cooling to <40 °C and mixing with an ultra-turrax.

### B) Measurements and Applications of Isosorbide Monoester (Sulfate)/Fatty Alcohol (Sulfate) Mixtures

### Example 4: Clear Melting Points Measurements

The clear melting points of isosorbide monoester, fatty alcohols and mixtures thereof were measured with a Reichert-Jung microscope Type 302101, using a Reichert Jung heating system Type 651501 and heating plate V40W80 R6416 with a testoterm 7000 pT 100 temperature sensing device while operating with polarized light. For the optical magnification to detect the clear melting point visually, a Bausch & Lomb WF10x/18 binocular and 60/-objective was used in the microscope. Before measurement the samples were heated 15 °C above the estimated melting point and a droplet of the sample was placed on a cover glass (VWR 18×18 mm No. 1 ECN 631-1567), covered with a plastic cap and stored for 24 h at 23 °C. The plastic cap was removed, and the cover glass was then placed on the heating plate. The heating system was started on lowest heat transfer rate. The sample was observed and the clear melting point in the display of the testoterm temperature sensing device noted once the test sample changes from non-transparent solid to clear liquid. The standard deviation of the clear melting point is ±0.5 °C.

The clear melting point measurements for the isosorbide monoester, fatty alcohols and mixtures thereof are shown in Table 1.

TABLE 1

| | | Mixture C = w/w % | Average Melting | |
|---|---|---|---|---|
| | | substance A:w/w % | Temperature of | |
| Substance A | Substance B | substance B | Mixture C | |
| C12 fatty alcohol | / | 100:0 | 23.5 | °C |
| C14 fatty alcohol | / | 100:0 | 29.5 | °C |
| C12/C14/C16/C18 | / | 100:0 | 23.5 | °C |
| fatty alcohol | | | | |
| C12/C14 fatty | / | 100:0 | 20 | °C |
| alcohol | | | | |
| C16/18 fatty | / | 100:0 | 53.5 | °C |
| alcohol | | | | |
| C12/C14 | / | 100:0 | 61.9 | °C |
| Isosorbide | | | | |
| monoester | | | | |
| C12/C14 | C12/C14 fatty | 30:70 | 36.6 | °C |
| Isosorbide | alcohol | | | |
| monoester | | | | |
| C12/C14 | C12/C14 fatty | 50:50 | 45.7 | °C |
| Isosorbide | alcohol | | | |
| monoester | | | | |
| C12/C14 | C12 fatty | 30:70 | 45.5 | °C |
| Isosorbide | alcohol | | | |
| monoester | | | | |
| C12/C14 | C12 fatty | 50:50 | 50.5 | °C |
| Isosorbide | alcohol | | | |
| monoester | | | | |
| C12/C14 | C12 fatty | 70:30 | 60 | °C |
| Isosorbide | alcohol | | | |
| monoester | | | | |
| C12/C14 | C14 fatty | 30:70 | 46 | °C |
| Isosorbide | alcohol | | | |
| monoester | | | | |
| C12/C14 | C14 fatty | 50:50 | 53.5 | °C |
| Isosorbide | alcohol | | | |
| monoester | | | | |
| C12/C14 | C14 fatty | 70:30 | 58.5 | °C |
| Isosorbide | alcohol | | | |
| monoester | | | | |
| C12/C14 | C16/18 fatty | 30:70 | 48 | °C |
| Isosorbide | alcohol | | | |
| monoester | | | | |
| C12/C14 | C16/18 fatty | 50:50 | 55 | °C |
| Isosorbide | alcohol | | | |
| monoester | | | | |
| C12/C14 | C16/18 fatty | 70:30 | 59 | °C |

| | |
|---|---|
| Isosorbide | alcohol |
| monoester | |

### Example 5: Viscosities

Oscillatory Temperature Sweep experiments were performed using a HR 20 Discovery Hybrid Rheometer (TA Instruments, Germany) at a constant strain amplitude (10%) and frequency (1 Hz) and using a plate-plate geometry (diameter=40 mm, gap=1000 μm). The materials were first subjected to a temperature ramp in the range of 30-100 °C or 40-100 °C with a rate of increase of 1 °C/min. The thermos-reversibility of the materials was then investigated by decreasing the temperature at the same rate (1 °C/min) from 100 °C to the starting temperature set for the ascending ramp.

Temperature dependent viscosities for i) C12/C14 isosorbide monoester, ii) 50:50 C12/C14 isosorbide monoester/C12/C14 fatty alcohol mixture and iii) 30:70 C12/C14 isosorbide monoester/C12/C14 fatty alcohol mixture are shown in FIG. 1.

### C) Example 6: Emulsion Polymerization of a Styrene/Acrylic Resin with a C12/C14 Isosorbide Monoester Sulfate/C12/C14 Fatty Alcohol Sulfate Mixture

### Equipment:

1.7 litre reaction vessel equipped with anchor stirrer (stirring speed 100 rpm) which was submersed into a water bath for temperature control, and feeding vessels (stirred for the monomer premix, non-stirred for the initiator feed) which were attached to weight modules for precise control of feeding.

### Preparation of Comonomer-Premix:

Styrene, n-butyl acrylate, methacrylic acid, demineralized water, and a C12/C14 isosorbide monoester sulfate/C12/C14 fatty alcohol sulfate mixture was mixed to yield the comonomer premix which was filled into the stirred feed vessel.

### Preparation of Initiator Solution:

Sodium metabisulfite was dissolved in demineralized water to give the initiator solution which is filled into a non-stirred feed vessel.

### Reaction Procedure:

The reaction vessel was pre-charged with a solution of potassium persulfate in demineralized water. Then, 225 g of the comonomer premix were added and temperature was raised to 60 °C. At 60 °C, 6 g of the initiator solution were added into the reaction vessel. After 5 min. the feeding of the comonomer premix and the initiator solution was started simultaneously. The comonomer premix was fed over a period of 150 minutes, the initiator solution over a period of 160 minutes. After the initiator solution had been fed completely, the temperature was raised to 65 °C and the mixture was stirred for another hour. The obtained dispersion was cooled to room temperature. The pH value was adjusted to 7-8 with aqueous ammonia solution (25% by weight).

### Work-Up and Analysis:

The above dispersion was filtered over a 125 μm filter. The filter residue was rinsed with water, dried and weighed to give the coagulum content.

The quantities of the reactants mentioned above are given in Table 2.

TABLE 2

| | | Example 6 A | Example 6 B |
|---|---|---|---|
| | | (inventive) | (comparative) |
| | Reactants | [in g] | [in g] |
| Reactor | Potassium persulfate | 4.00 | 4.00 |
| precharge | DI water | 250.00 | 250.00 |
| Feed 1 | Styrene | 400.00 | 400.00 |
| | Butyl acrylate | 400.00 | 400.00 |
| | Methacrylic acid | 15.00 | 15.00 |
| Preemulsion | Emulsifier, C12/C14 | 20.00 | — |
| | isosorbide monoester | | |
| | sulfate/C12/C14 fatty | | |
| | alcohol sulfate mixture | | |
| | (active matter) | | |
| | Disponil@ FES 27 | — | 20.00 |
| | Fatty alcohol ether | | |
| | sufalte (2EO), Na-salt | | |
| | DI water | 450.00 | 450.00 |
| Feed 2 | Sodium metabisulfite | 4.00 | 4.00 |
| Initiator | DI water | 100.00 | 100.00 |

The styrene/acrylic dispersions prepared in example 6A and 6B were analyzed. The analytical data is specified in Table 3.

### Analytical Methods for the Polymer Dispersions:

Solid Content: Determined by drying about 5 g of polymer dispersion at 150 °C for 20 minutes using a Mettler Toledo Moisture Analyzer HR 83.

Viscosity: Determined at room temperature by using a Brookfield viscometer DV-II+ (RV spindle set) at 20 rpm.

Conversion: Calculated by dividing the measured solid content by the theoretical solid content.

Coagulum: Reported as dry coagulum, as the sum of the coagulum obtained by filtering the polymer dispersion using a 125-micrometer filter and the coagulum that is collected from the stirrer and reactor wall. The wet coagulum is dried in the Mettler Toledo Moisture Analyzer HR 83 at 130 °C until the weight is constant.

Particle size: Is reported as the d50 measured with a Beckman Coulter LS 13320 Laser Diffraction Particle Sizer Analyzer.

Visual evaluation: The polymer dispersion is casted on a glass plate with a 100-micrometer knife coater. After drying 24 hours at room temperature, the polymer film is evaluated visually and graded on a scale of 1 to 6, 1 is for the best and 6 is for the worst.

Water absorption: A polymer film (approx. 75 mm×35 mm×2 mm) is cast by drying the polymer dispersion in a teflon form for 48 hours at 50 °C. The dried film is weighed and then submerged into deionized water at room temperature for 24 hours. Then the film weighed again after removing excess water. The weight increase due to absorption of water is reported in percentage.

Electrolyte stability: 6 electrolyte solutions are prepared and tested in this order: 1% NaCl, 10% NaCl, 1% CaCl2, 10% CaCl2, 1% Al2(SO4)3, 10% A12(SO4)3. To check the electrolyte stability 10 ml of polymer dispersion is mixed with 10 ml of electrolyte solution. It is visually observed if the polymer dispersion coagulates after shaking of the mixture. The electrolyte concentration where the polymer dispersion is still stable (does not coagulate) is reported.

Shear stability: 50 g polymer emulsion is filled into a glass cylinder and put under shear stress using a high-speed stirrer (Company LatexMST, Model MK2 for synthetic rubber) at 14,000 rpm. The shear stress is applied for 30 minutes. If the emulsion coagulates before 30 minutes, the time at which the coagulation occurs is recorded. If the emulsion is stable for 30 minutes, it is filtered, and the filter residue is determined.

TABLE 3

| | Dispersion from | Dispersion from |
|---|---|---|
| | Example 6A | Example 6B |
| | Inventive example | Comparative example |
| Surfactant used: | C12/C14 isosorbide | Disponil@ FES 27 |
| | monoester sulfate/ | Fatty alcohol ether |
| | C12/C14 fatty alcohol | sufalte (2EO), Na-salt |
| | sulfate mixture | |
| Solid content [%] | 51.21 | 50.6 |
| Viscosity [mPas] | 88 | 230 |
| Conversion [%] | 100 | 99 |
| Coagulum [%] | 0.2 | 0.2 |
| Particle Size [nm] | 171 | 102 |
| Visual evaluation | 1 | 1 |
| [scale 1-6] | | |
| Water absorption [%] | 3.2 | 4.0 |
| Electrolyte stability | 1% NaCl | 1% NaCl |
| Shear stability | Stable | Stable |

From Table 3 can be seen that a stable styrene/acrylic polymer can be prepared by using an anionic surfactant mixture of the present invention as an emulsifier. The conversion and water absorption are improved compared to the values of the comparative example.

1.-16. (canceled)

17. A method for preparing isosorbide ester sulfate comprising the steps a) to c) as follows:
a) reaction of i) isosorbide and ii) carboxylic acid comprising at least 10 carbon atoms, in order to obtain a first intermediate (I1);
b) mixing i) the first intermediate (I1) with ii) fatty alcohol comprising at least 10 carbon atoms, in order to obtain a second intermediate mixture (I2), wherein the second intermediate mixture (I2) comprises 35 to 90% by weight of the fatty alcohol; and
c) reaction of i) the second intermediate mixture (I2) with ii) at least one of sulfur trioxide, oleum or disulfuric acid in a falling film reactor,
in order to obtain the isosorbide ester sulfate.

18. The method according to claim 17, wherein the carboxylic acid comprising at least 10 carbon atoms
is linear;
has as the only functional group one carboxyl group; and/or
is a C12 or C14 carboxylic acid or a mixture thereof.

19. The method according to claim 17, wherein the fatty alcohol comprising at least 10 carbon atoms
is linear;
has as the only functional group one primary alcohol group; and/or
is a C12, C14, C16 or C18 fatty alcohol or a mixture thereof.

20. The method according to claim 17, wherein the second intermediate mixture (I2) comprises 40 to 85% by weight of the fatty alcohol or its mixtures.

21. The method according to claim 17, wherein in step c) the second intermediate mixture (I2) is reacted with sulfur trioxide.

22. The method according to claim 17, wherein the temperature in step c) is not higher than 98 °C.

23. The method according to claim 17, wherein in step a) the molar ratio of isosorbide to carboxylic acid is at least 1.5:1.

24. The method according to claim 17, wherein the first intermediate (I1) is purified by distillation.

25. Use of fatty alcohol comprising at least 10 carbon atoms during sulfation of isosorbide ester for lowering the melting temperature and/or viscosity of the isosorbide ester, wherein the isosorbide ester comprises an alkyl group possessing at least 10 carbon atoms and the sulfation is conducted in a falling film reactor.

26. A composition comprising an isosorbide ester sulfate and a fatty alcohol sulfate, wherein the isosorbide ester sulfate and the fatty alcohol sulfate each comprise an alkyl group possessing of at least 10 carbon atoms and at least 40% of the combined weight of the isosorbide ester sulfate and the fatty alcohol sulfate is based on the fatty alcohol sulfate.

27. The composition according to claim 26, wherein the alkyl group of the isosorbide ester sulfate
is linear; and/or
is a C12 or C14 alkyl group or the composition comprises a mixture of C12 and C14 isosorbide ester sulfates.

28. The composition according to claim 26, wherein the fatty alcohol sulfate
is linear; and/or
is a C12, C14, C16 or C18 fatty alcohol sulfate or a mixture thereof.

29. The composition according to claim 26, wherein at least 45% of the combined weight of the isosorbide ester sulfate and the fatty alcohol sulfate is based on the fatty alcohol sulfate or its mixtures.

30. The composition according to claim 26, wherein said composition is selected from the group consisting of cleaning composition, fabric and home care product, industrial and institutional cleaning product, cosmetic formulation, emulsifier for emulsion polymerization, crude oil emulsion breaker, pigment dispersion for ink jet inks, formulation for electro plating, cementitious composition, and dispersant for agrochemical formulations.

31. The composition according to claim 26 further comprising:
a) at least one element of the group consisting of non-anionic surfactants, amphoteric surfactants, builders, chelating agents, co-builders, alcohols, thickeners, water soluble polymers, clay soil removal/anti-redeposition agents, polymeric soil release agents, bleaching agents, bleach activators, brighteners, malodor control agents, pigments, dyes, opacifiers, hueing agents, dye transfer inhibiting agents, suds suppressors (antifoams), anti-corrosion agents, softeners and perfumes;
b) an antimicrobial agent selected from the group consisting of 2-phenoxyethanol and 4,4′-dichloro 2-hydroxydiphenylether; and/or
c) at least one enzyme selected from the list consisting of lipases, hydrolases, amylases, DNases, proteases, cellulases, hemicellulases, phospholipases, esterases, mannanases, xylanases, dispersins, oxidoreductases, cutinases, pectate lyases, pectinases, lactases and peroxidases, and combinations of at least two of the foregoing types.

32. Use of a composition according to claim 26,
A) a cleaning composition and/or fabric and home care product, for
i) improved removal of oily/fatty stains, and/or
ii) clay removal, and/or
iii) soil removal of particulate stains, and/or
iv) dispersion and/or emulsification of soils, and/or
v) modification of treated surface to improve removal upon later re-soiling, and/or
vi) whiteness improvement and/or
i) removal of oily/fatty stains and/or ii) clay removal,
each of the before mentioned options i) to vi) for use in a laundry detergent formulation and/or a manual dish wash detergent formulation and/or in a formulation suitable for (pre)-treatment of textiles and/or soap bars; or
B) as an emulsifier in a polymerization reaction.
